(19) United States
(12) Patent Application Publication
(10) Pub. No.: US 2022/0378826 A1
(43) Pub. Date: Dec. 1, 2022

(54) TUMOUR INFILTRATING LYMPHOCYTE THERAPY AND USES THEREOF

(71) Applicant: Instil Bio (UK) Limited, Manchester (GB)

(72) Inventors: Nicola Kaye Price, Manchester (GB); John Stephen Bridgeman, Manchester (GB)

(21) Appl. No.: 17/570,556

(22) Filed: Jan. 7, 2022

Related U.S. Application Data
(63) Continuation-in-part of application No. PCT/GB2020/051790, filed on Jul. 24, 2020.
(60) Provisional application No. 62/878,001, filed on Jul. 24, 2019.

(30) Foreign Application Priority Data
Jul. 24, 2019 (GB) 1910605.3

(51) Int. Cl. A61K 35/17; A61K 38/17; A61P 35/00; C12N 5/0783; G01N 33/50

(52) U.S. Cl. CPC A61K 35/17; A61K 38/1774; A61P 35/00; C12N 5/0636; G01N 33/5091; G01N 33/505; C12N 2501/2302; C12N 2501/2304; C12N 2501/2312; C12N 2501/998

(57) ABSTRACT

The present invention concerns a biomarker useful in adoptive cell therapy. The biomarker in question is CD150, otherwise termed SLAM or SLAMF1. Herein Applicants demonstrate that expression of CD150 on tumour infiltrating lymphocytes infusion products correlates with the response rate seen in those patients. High CD150 expression is found on patients who go on to have a complete response and low expression on patients who do not respond to therapy. The invention relates to the use of the biomarker to predict response rate or stratify patients for treatment. It also covers exploitation of this receptor in adoptive cell therapy regimens in general, including but not limited to over expression of the receptor in T-cell populations or isolation of cells expressing CD150 in an effort to increase efficacy.

### RELATED APPLICATIONS AND INCORPORATION BY REFERENCE

This application is a continuation-in-part of international application PCT/GB2020/051790 filed Jul. 24, 2020 and published as international publication WO2021/014174 on Jan. 28, 2021 and which claims priority to GB patent application Serial No GB1910605.3, filed Jul. 24, 2019, and U.S. provisional application Ser. No. 62/878,001, filed Jul. 24, 2019, each incorporated by reference herein in its entirety.

Reference is made to international patent application Serial No. PCT/GB2019/050188, filed Jan. 23, 2019 and GB1801067.8, filed Jan. 23, 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to the field of prognosis of cancer following treatment with T-cells including Tumour Infiltrating Lymphocytes (TILs). The prognosis is based on the quantification of a biological marker expressed by the T-cells including Tumour Infiltrating Lymphocytes. The invention also relates to the exploitation and/or manipulation of the biological markers to enhance the therapeutic efficacy of T-cell therapies including TIL therapy.

### BACKGROUND OF THE INVENTION

Adoptive cell therapy (ACT) using autologous T-cells to mediate cancer regression has shown much promise in early clinical trials. Several general approaches have been taken such as the use of naturally occurring tumour reactive or tumour infiltrating lymphocytes/Tumour associated lymphocytes (TILs) expanded ex vivo. Additionally, T-cells may be modified genetically to retarget them towards defined tumour antigens. This can be achieved via the gene transfer of: peptide (p)-major histocompatibility complex (MHC) specific T-cell Receptors (TCRs); or synthetic fusions between tumour specific single chain antibody fragment (scFv) and T-cell signalling domains (e.g. CD3ζ), the latter being termed chimeric antigen receptors (CARs). TIL and TCR transfer has proven particularly effective when targeting Melanoma (Rosenberg et al., 2011; Morgan et al., 2006), whereas CAR therapy has shown much promise in the treatment of certain B-cell malignancies (Grupp et al., 2013).

Tumour Infiltrating Lymphocyte therapy has been applied to a number of different malignancies including gastric (Xu et al., 1995), renal (Figlin et al., 1997; Goedegebuure et al., 1995), cervical (Stevanovic et al., 2015) and colorectal cancers (Gardini et al., 2004), but has been most widely applied and shown most development and promise in melanoma therapy. Trials in advanced metastatic melanoma have consistently shown around 50% response rate with 15-20% complete response (cure) (Rosenberg et al., 2011; Dudley et al., 2010). Additionally, tumour associated lymphocytes can be obtained from ascites and grown in the same manner as TIL.

The process of TIL production offers lower costs and improved technological innovations compared to gene-modified T-cells; however, the process is more laborious and requires a higher degree of user skill to optimise growth. In current methodologies, tumour biopsies are taken from the patient and transferred to a laboratory setting. There are two options for TIL production: i) the tumour is cut into small fragments approx. 1-2 mm³ and seeded in individual wells of 24-well tissue culture plates; or ii) the tumour is enzymatically digested and the resulting single cell suspension is cultured in 24-well tissue culture plates. In both cases the TIL are then cultured for 2-3 weeks with >3000 IU/ml IL-2, after which they undergo a two week expansion with irradiated feeder cells to obtain generally >1×10¹⁰ cells for infusion. During the expansion phase, the patient receives preconditioning chemotherapy (typically cyclophosphamide and fludarabine), and upon infusion of the cells receives supportive IL-2 treatment to enhance TIL engraftment.

With any therapeutic intervention there is a mixed patient response and as part of the refinement of the treatment the goal is then to determine which patients will show a clear benefit from the treatment given. Immunotherapeutics are no exception and as classical examples the pre-treatment mean corpuscular hemoglobin concentration has been shown to predict the outcome of TroVax vaccination (Harrop et al., 2012) and PDL1 expression has been used to define the patients who show more benefit from treatment with anti-PD1 therapy (Topalian et al. 2012). As TIL therapy in melanoma has around a 50% response rate, it would be beneficial to find markers which may predict those patients who will benefit most from treatment, particularly as agents used alongside the TIL are potentially very toxic (IL-2 and preconditioning chemotherapy). To this end it has been suggested that TIL products enriched in effector memory T-cells demonstrate better patient responses (Radvanyi et al., 2015). Furthermore, BTLA expression in TIL has been correlated with a good prognosis following TIL infusion (Radvanyi et al., 2012; Haymaker et al., 2015).

Mehrle et al, 2008 describes the effect of modulating both CD150 and SAP in activated lymphocytes by gene silencing or overexpression. Mehrle does not describe the modulation of CD150 expression in tumor reactive T cells using cytokines.

Browning at al, 2004 describes CD150 as a marker for alloactivated CD4+25+ for use in a setting where there is on-going alloreactivity (e.g. graft versus host disease) or autoimmunity.

WO2017/179015 discloses chimeric antigen receptors with a target binding domain capable of selectively binding placenta-like chondroitin sulfate A (pl-CSA) and does not relate to methods for preparing a population of tumor reactive T cells.

Citation or identification of any document in this application is not an admission that such document is available as prior art to the present invention.

### SUMMARY OF THE INVENTION

The invention provided herein relates to a cell surface marker which correlates with successful treatment following infusion and thus could be used as a marker of prognosis as TIL populations expressing this marker appear to show increased tumour reactivity as they are associated with improved clinical response rates.

The invention also describes how this receptor can be exploited to improve TIL and other T-cell therapies including therapies that utilise gene-modified T-cells.

The present inventors have identified a cell surface marker: Signaling lymphocytic activation molecule (also known as SLAM/SLAMF1/SLAM family member 1/CD150/CDw150/IPO-3; Human amino acid sequence, see: NCBI Reference Sequence: NP_003028.1; the different terms for CD150, e.g. SLAM/SLAMF1 are used interchangeably herein), which correlates with successful treatment following T-cell infusion (TIL infusion), thus the marker indicates that the T-cells (for example TILs) are likely to be tumour reactive. As used herein, the term tumor reactive T cells refers to T cells that carry a T-cell receptor (TCR) specific to an antigen on a tumour. For example, the T cells have cytotoxic activity against tumour cells. At present it is not known by what mechanism this increased tumour reactivity occurs, for example, it may be via enhanced tumour cell killing in the SLAM expressing population and/or, for example, SLAM expression could improve cell survival and persistence in the tumour microenvironment. The present invention relates to how this receptor can be exploited to improve TIL therapy and other cancer therapies that use T-cells, including therapies that utilise gene modified T-cells.

In a first aspect, provided herein is a method for obtaining a cell population, such as a T cell population, which is enriched for tumour reactive T-cells, wherein T-cells expressing CD150/SLAM/SLAMF1 are selected and optionally expanded. Thus cells expressing CD150/SLAM/SLAMF1 may be selected from a bulk population of cells, such as a T-cell population (e.g. gene modified T-cells) or a T cell population which includes a small proportion of other cell types (e.g. a TIL population).

The T-cells expressing CD150/SLAM/SLAMF1 may be selected from cells originating from a patient (e.g. TIL cells from a tumour biopsy, lymph nodes, ascites). In embodiments, the selection of T-cells expressing CD150/SLAM/SLAMF1 comprises one or more of (i) flow cytometry, (ii) antibody panning, (iii) magnetic selection, (iv) biomarker targeted cell enrichment. The selection may comprise contacting the cell population with an anti-CD150/SLAM/SLAMF1 antibody. The selected cells may then be separated and expanded to enrich the number of CD150/SLAM/SLAMF1 positive (+ve) cells present in the population.

In embodiments provided herein, TILs expressing the biological marker CD150/SLAM/SLAMF1 are expanded by way of one or both of the following options:

a. Expansion with irradiated feeder cells to provide a T-cell activation signal and costimulation driven by antibodies or costimulatory receptors; and/or

b. Expansion with immobilised or soluble reagents which provide a T-cell activation signal and costimulation signals driven through said one or more biological markers.

In embodiments provided herein, the cell population is selected from: i) a population of tumour infiltrating lymphocytes (TILs) from a tumour biopsy, lymph node or ascites; and/or ii) a population of gene modified T-cells for example T-cells engineered to express a CAR and/or a TCR and/or other exogenous nucleic acid.

In another aspect of the invention provided herein is a population of cells enriched for tumour reactive T-cells which has been obtained according to any of the methods provided herein. Such a population may have been obtained by starting with a TIL population or a population gene modified T-cells and selected and optionally enriching for CD150/SLAM/SLAMF1 positive (+ve) cells in that population, for example using any of the methods provided herein.

Also provided herein is a population of cells enriched for tumour reactive T-cells (such as TILs or gene modified T-cells), wherein >25% of the T-cells or TILs express biological marker CD150/SLAM/SLAMF1, or wherein >30%, >35% or >40% of the T-cells or TILs express biological marker CD150/SLAM/SLAMF1.

In embodiments, provided herein is a population of TILs enriched for biological marker CD150/SLAM/SLAMF1. In one embodiment, the population of TILs enriched for biological marker CD150/SLAM/SLAMF1 is obtained according to a method described herein. In some embodiments, the TILs may originate from a melanoma.

T cells (including T-cells obtained from a TIL population) may be engineered to express or over-express CD150/SLAM/SLAMF1 from an exogenous nucleic acid. Expressing or over-expressing CD150/SLAM/SLAMF1 in such a manner may increase the tumour reactivity of the engineered T cell. In one embodiment, provided herein is a T-cell comprising a first exogenous nucleic acid encoding CD150/SLAM/SLAMF1. The T-cell may further comprise a second exogenous nucleic acid encoding a Chimeric Antigen Receptor (CAR) and/or T-cell receptor (TCR) and/or other protein.

Suitably, the T-cell is a CD4+ or CD8+ cell. In some embodiments, the T-cell is a memory cell.

In certain embodiments, expansion of a T cell population comprises providing conditions that favor expansion of particular subpopulations, more particularly subpopulations of cells that express CD150/SLAM/SLAMF1. In certain embodiments, expansion of central memory is favored. In an embodiment, expansion of central memory favors or comprises expansion of central memory CD4+ cells. In an embodiment, expansion of central memory favors or comprises expansion of central memory CD8+ cells. A non-limiting measure of central memory is the proportion of expanded cells that expresses CD62L+/CD45RO+. In an embodiment, expansion of effector memory is favored. In an embodiment, expansion of effector memory favors or comprises expansion of effector memory CD4+ cells. In an embodiment, expansion of effector memory favors or comprises expansion of effector memory CD8+ cells. A non-limiting measure of effector memory is the proportion of expanded cells that expresses CD62L−/CD45RO+.

In certain embodiments, expansion of a T cell population comprises providing conditions that modulate effector function. In certain embodiments, IL-2 and IL-12 are provided and the expanded T cell population comprises an increased proportion of CD4+ cells and/or CD4+/CD8+ cell compared to IL-2 alone. In certain embodiments, IL-7 and/or IL-15 is provided with IL-2 and IL-12 and the expanded T cell population comprises an increased proportion of CD4+ cells and/or CD4+/CD8+ cells compared to IL-2 alone.

In certain embodiments, IL-2 and IL-12 are provided and the expanded T cell population comprises an increased proportion of central memory T cells. In certain such embodiments, the proportion of effector memory cells is reduced. In certain embodiments, IL-7 and/or IL-15 is provided with IL-2 and IL-12 and the expanded T cell population comprises an increased proportion of central memory T cells. In certain such embodiments, the proportion of effector memory cells is reduced.

In certain embodiments, IL-2 and IL-12 are provided and the expanded T cell population comprises an increased proportion of CD8+ cells that express IFNγ. In certain embodiments, IL-7 and/or IL-15 is provided with IL-2 and IL-12 and the expanded T cell population comprises an increased proportion of CD8+ cells that express IFNγ. In certain embodiments, IL-2 and IL-12 are provided and the expanded T cell population comprises an increased proportion of CD8+ cells that express TNFα. In certain embodiments, IL-7 and/or IL-15 is provided with IL-2 and IL-12 and the expanded T cell population comprises an increased proportion of CD8+ cells that express TNFα.

In certain embodiments, T cell populations are expanded with combinations of cytokines, including without limitation, IL-7+IL-15, IL-2+IL-7+IL-15, IL-2+IL-12, IL-2+IL-18, IL-2+IL-12+IL-7+IL-15, IL-2+IL-12+IL-7+IL-15+IL-6, IL-2+IL-12+IL-7+IL-15+IL21, IL-2+IL-12+IL-7+IL-15+IL-6+IL-21, IL-7+IL-15+IL-6, IL-7+IL-15+IL-21, IL-7+IL-15+IL-6+IL-21, IL-2+IL-12+IL-6, IL-2+IL-12+IL-21, or IL-2+IL-12+IL-6+IL-21.

In certain embodiments, T cell population are expanded with a Th2 blocking reagent, such as, without limitation, αIL-4. Non-limiting examples include IL-7+IL-15+αIL-4, IL-2+IL-7+IL-15+αIL-4, IL-2+IL-12+αIL-4, IL-2+IL-18+αIL-4, IL-2+IL-12+IL-7+IL-15+αIL-4, IL-2+IL-12+IL-7+IL-15+IL-6+αIL-4, IL-2+IL-12+IL-7+IL-15+IL21+αIL-4, IL-2+IL-12+IL-7+IL-15+IL-6+IL-21+αIL-4, IL-7+IL-15+IL-6, IL-7+IL-15+IL-21+αIL-4, IL-7+IL-15+IL-6+IL-21+αIL-4, IL-2+IL-12+IL-6+αIL-4, IL-2+IL-12+IL-21+αIL-4, or IL-2+IL-12+IL-6+IL-21+αIL-4.

In certain embodiments wherein there is a combination of cytokines that includes IL-12 and other cytokines, the IL-12 is present in an initial portion of the expansion period, but not in a later portion of the expansion period. For example, in certain embodiments wherein there is cytokine combination that includes IL-2, IL-12, IL-7, and IL-15, the IL-12 can be initially present and then switched out. In certain such embodiments, the IL-7 and/or IL-15 is added when IL-12 is switched out. In certain such embodiments, the IL-7 and/or IL-15 is present throughout the expansion.

In an embodiments, a expanded cell population of the invention suitable for use in therapy comprises 10⁹ of more cells. In another embodiment, a expanded cell population of the invention suitable for use in therapy comprises 5×10⁹ of more cells. In another embodiment, an expanded cell population of the invention suitable for use in therapy comprises 10¹⁰ of more cells.

In another aspect of the invention, provided herein is a method of treating a disease in a subject comprising administering T-cells, such as TILs or gene modified T-cells, that express CD150/SLAM/SLAMF1 to the subject. Also provided is an enriched and expanded cell population of tumour reactive T-cells for use in the treatment of cancer, comprising administering to a cancer patient in need thereof cells from a cell population, wherein the cell population is prepared by a method comprising identifying and/or obtaining a cell population expressing CD150/SLAM/SLAMF1 and expanding the cell population. The TILs may be a population of TILs enriched for CD150/SLAM/SLAMF1 by selection and expansion of cells originating from a subject or the TILs may be TILs or other T-cells that have been engineered to express CD150/SLAM/SLAMF1. The subject is typically a human. The method of treating is suitably adoptive cell therapy; T-cells may be autologous or allogenic. The disease is suitably cancer, e.g. melanoma, or ovarian or cervical cancer.

A further aspect provides a pharmaceutical composition comprising the isolated immune cell or the cell population as taught herein.

Also provided herein are pharmaceutical compositions suitable for intravenous infusion comprising a population of cells enriched for tumour reactive T-cells expressing CD150/SLAM/SLAMF1 including T-cells engineered to express CD150/SLAM/SLAMF1 together with a pharmaceutically acceptable carrier, diluent or excipient, and optionally one or more further pharmaceutically active polypeptides and/or compounds.

Also provided herein is a method for assessing the tumour reactivity of a cell population which method comprises quantifying T-cells in the cell population that are expressing SLAM/SLAMF1/CD150. Suitably, the cell population may be i) TILs from a patient and the T-cells expressing SLAM/SLAMF1/CD150 are quantified pre-REP and/or post-REP; or ii) a population of T-cells engineered to express an exogenous CAR and/or a TCR. When assessing SLAM/SLAMF1/CD150 expression levels, at least 25% of T-cells expressing SLAM/SLAMF1/CD150 in a population of T-cells/TILs indicates that the cell population is tumour reactive.

Aspects and embodiments of the invention are also described below.

The invention described herein relates to an in vitro method for the prognosis of patients who may receive Tumour Infiltrating Lymphocyte (TIL) therapy for cancer and exploitation of these prognostic markers to develop novel methods to generate a more optimal TIL product; the method comprises the following:—

i) Quantifying in a sample of tumour digest or TIL product during manufacture, from said patient, at least one biological marker (SLAM/CD150) on the TIL wherein the quantification is the proportion of cells expressing said biological marker (SLAM/CD150).

ii) Comparing the value obtained at step i) for said at least one biological marker with a predetermined reference value for the same biological marker; which predetermined reference value is correlated with a specific prognosis of progression of said cancer.

iii) Isolating cells from bulk TIL populations prior to, during or after manufacture based on enrichment of cells expressing the said biomarker(s).

iv) Overexpressing said biological marker in T-cells to improve the therapeutic efficacy of the product.

Also envisaged is the use of said at least one biological marker on a TIL as a release test for a TIL product.

Further provided are methods of manipulating T-cell populations to increase, enhance, or induce expression of said biological marker using chemicals, antibodies, other cells, proteins, lipids, or other undefined mechanisms or methods.

In some embodiments of the method, step i) consists of quantifying one or more biological markers by flow cytometry.

In some other embodiments of the method, step i) consists of quantifying said biological marker by gene expression analysis in the whole tumour tissue sample.

In some other embodiments of the method, step i) consists of quantification of the said biological marker by immunohistochemistry of the entire tumour tissue sample.

In some embodiments of the method, step iii) consists of isolating cells expressing the biomarker(s) using flow cytometric sorting.

In some other embodiments of the method, step iii) consists of isolating cells expressing the biomarker(s) using some other form of physical separation technique which may include but is not limited to: Miltenyi MACS separation, StemCell Technologies magnetic separation technology or flow cytometric sorting.

In some other embodiments of the method, step iii) consists of enriching cells expressing the biomarker(s) via some form of process of mitogenic stimulation such as by using antibodies or soluble receptor protein which engages this Biomarker in combination with stimulation of T-cell activation such as to induce costimulation though the biomarker.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1—TIL manufacturing process. Current TIL therapy is critically dependent on two distinct sites. In the clinical site the tumour is resected and sent to the second site (the manufacturing site) where the tumour is dissociated and the T-cells grown out using IL-2 in plates. After approximately two weeks the cells are put into a rapid expansion protocol (REP) to grow the cells to >1×10¹⁰ in number. During the REP the patient undergoes preconditioning chemotherapy. Post-REP The TIL final product is returned to the patient along with intravenous IL-2 to support the reinfused T-cells.

FIG. 2—Exemplary flow cytometric staining gating strategy for SLAM measurement. Pre or Post-REP TIL were stained with the following antibodies:

Fixable Viability Dye eFluor 450, αCD45RO FITC, αCD8 PE Vio770, αCD4 APC Cy7, αCD62L APC; and then counterstained with either pairs of mIgG1 PE and αSLAM PE. Cells were acquired on a MACSQuant analyser. Analysis was performed using MACSQuantify software using the gating strategies shown.

FIG. 3A—Expression of SLAMICD150 in TIL pre- and post-rapid expansion protocol (REP). Post-REP TIL were stained with the following:

Fixable Viability Dye eFluor 450, αCD45RO FITC, αCD8 PE Vio770, αCD4 APC Cy7, αCD62L APC; and then counterstained with either mIgG1 PE or αSLAM PE. Cells were acquired on a MACSQuant analyser. Analysis was performed using MACSQuantify software. The proportion of SLAM⁺ cells were determined in each CD4+ or CD8+ population and plotted using Graphpad software. For all three graphs, patients are stratified by clinical response (progressive disease, stable disease and responders), Response rates indicated are the best response achieved by the patient as determined by response evaluation criteria in solid tumours (RECISTv 1.1) measurements (refer to Schwartz et al. Eur J Cancer 2016) * P<0.05. For all three graphs shown the mean+/−SEM is plotted for each cell and patient subtype. Significance determined using 2-way ANOVA, followed by Sidak's multiple comparison test. For the top graph (Final product (CD4+)), Memory: PD vs R, **p=0.009. For the bottom graph (Final Product), CD4: PD vs SD, *p=0.037; PD vs R, **p=0.004; CD8: PD vs R, **p=0.008.

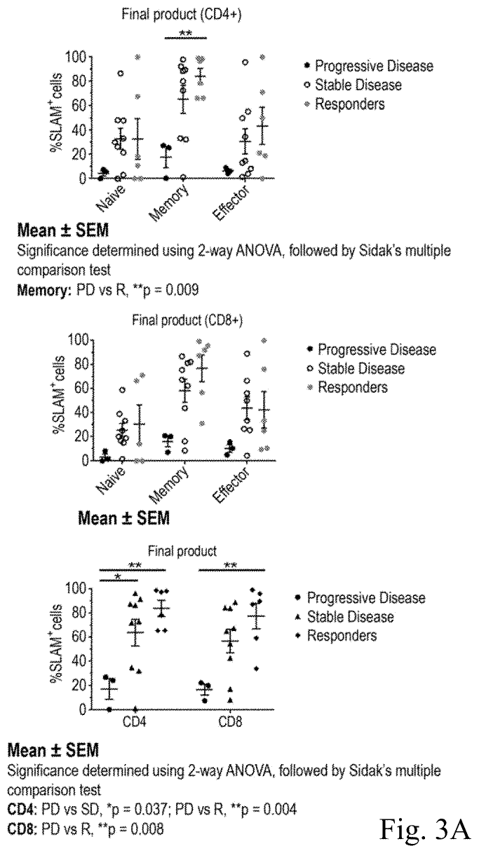

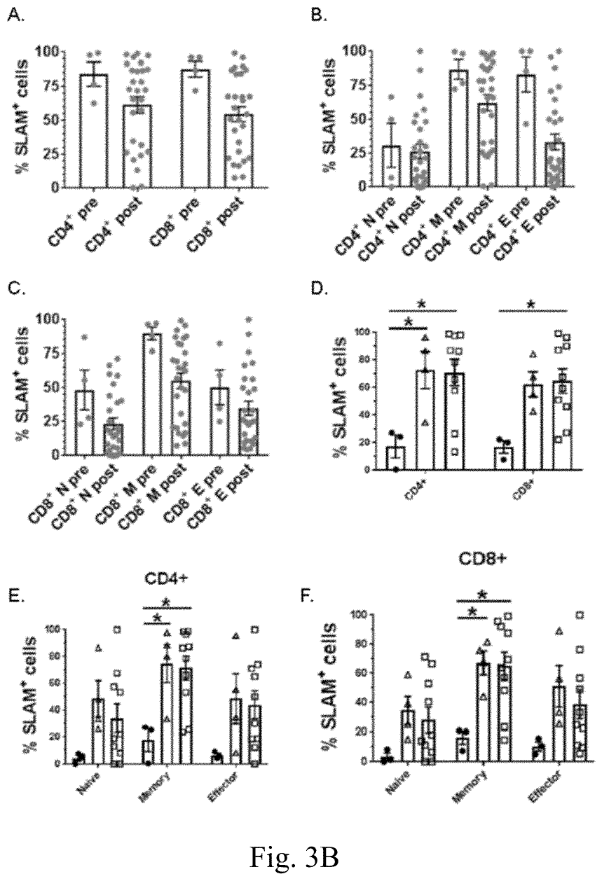

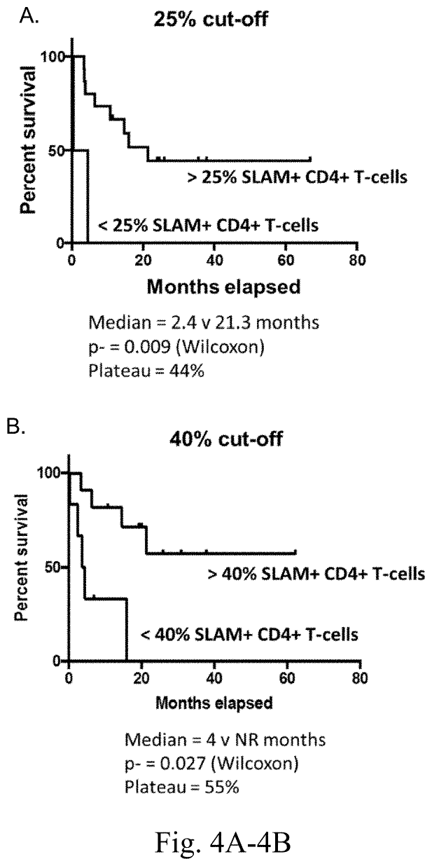

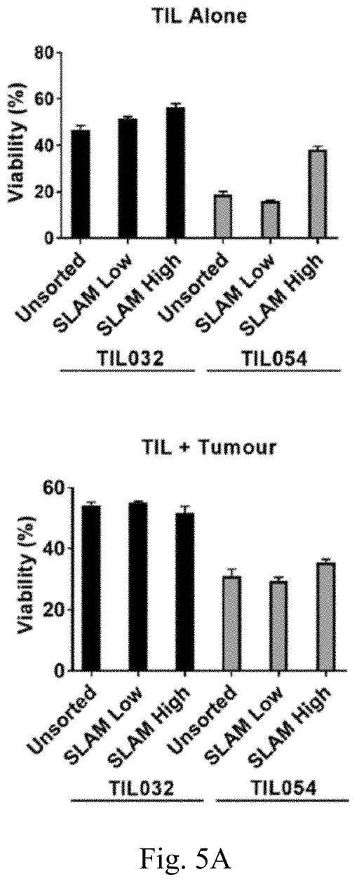

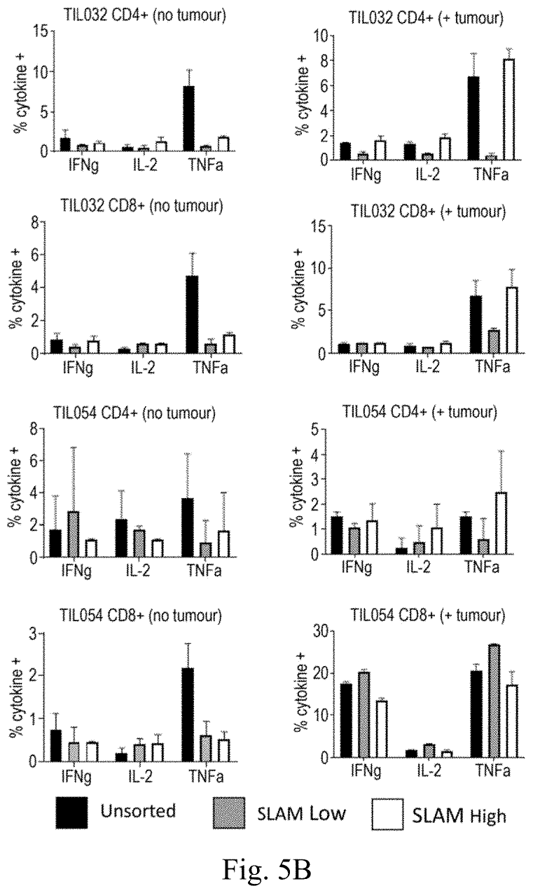

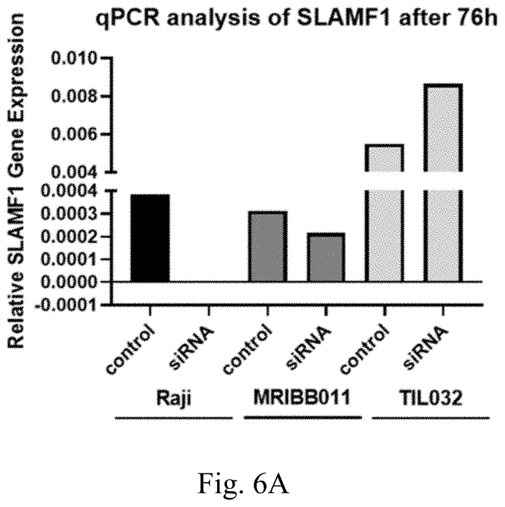

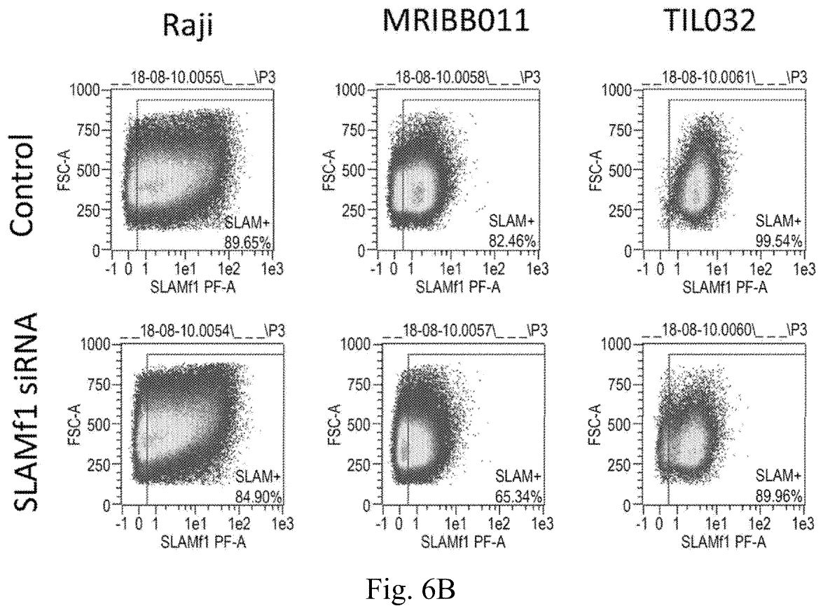

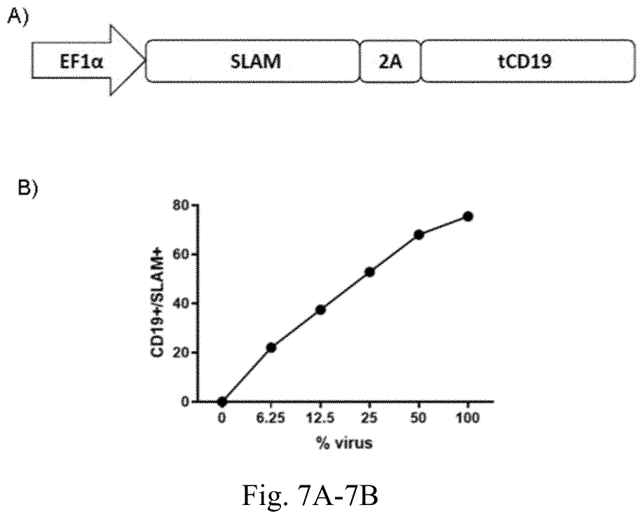

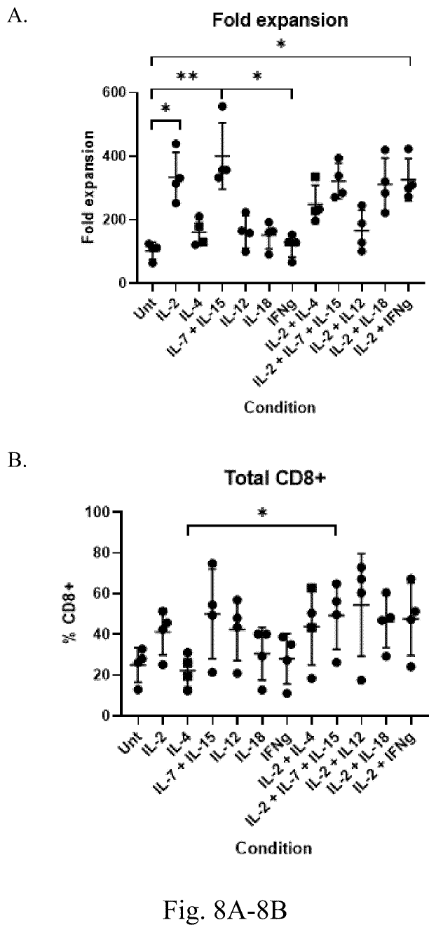

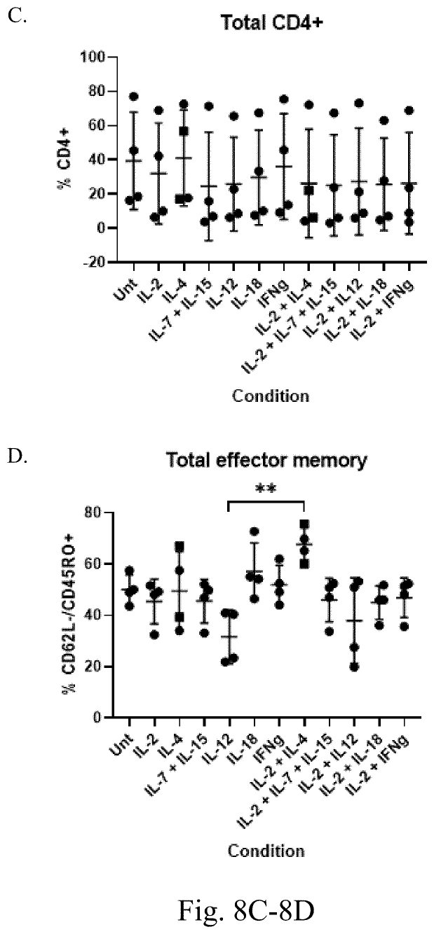

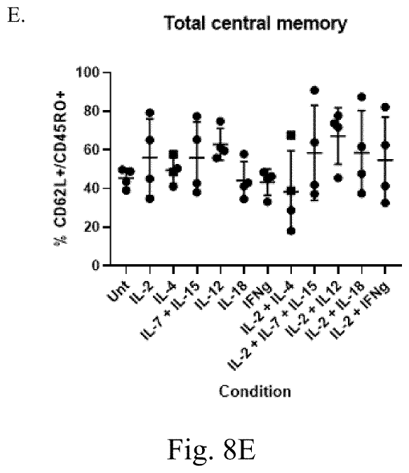

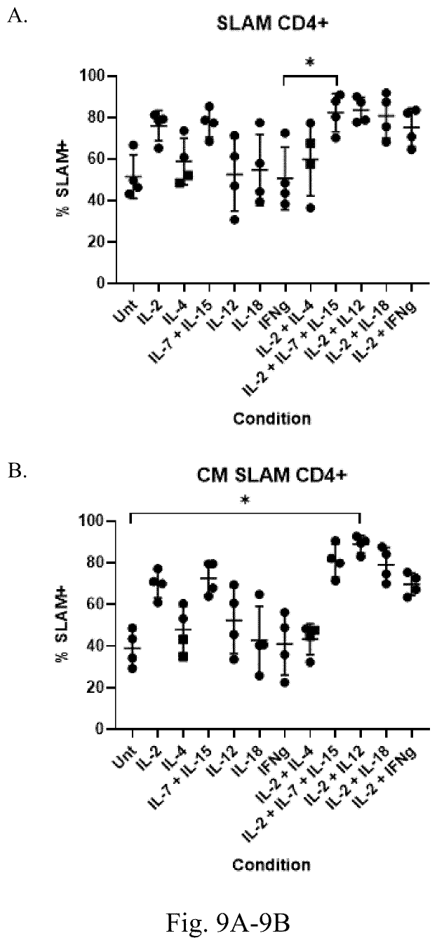

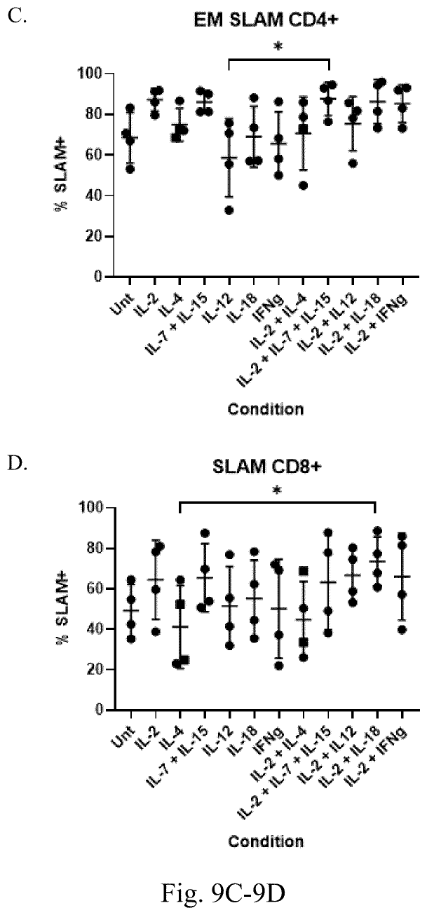

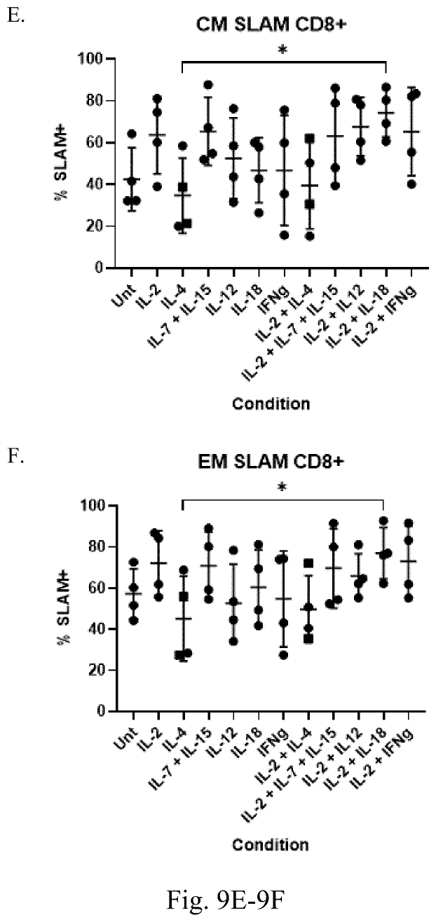

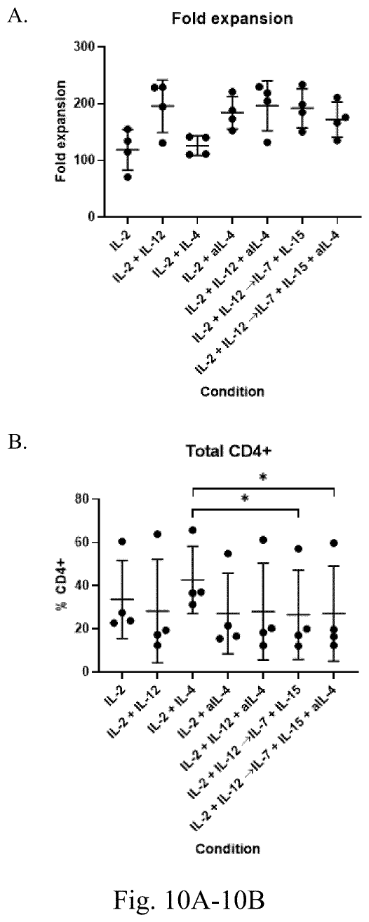

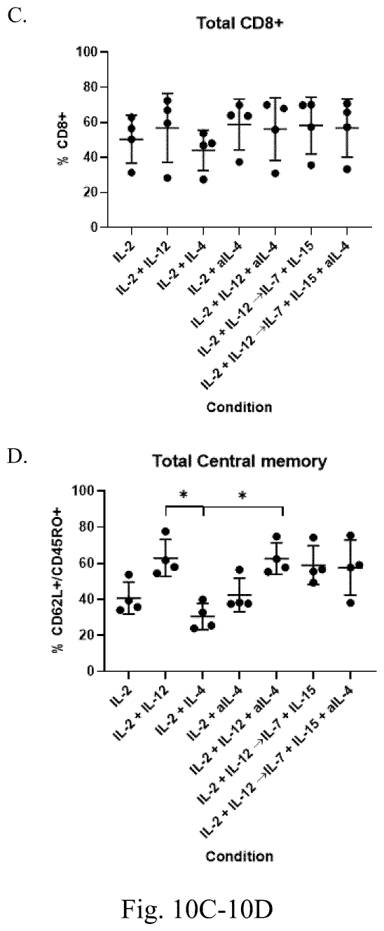

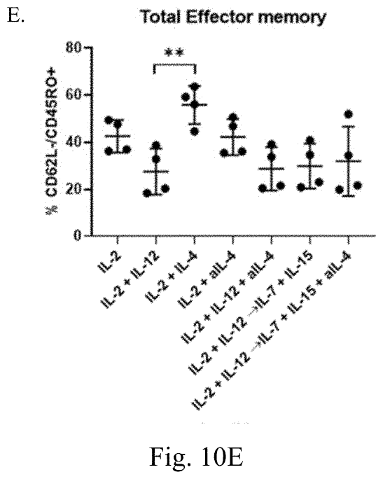

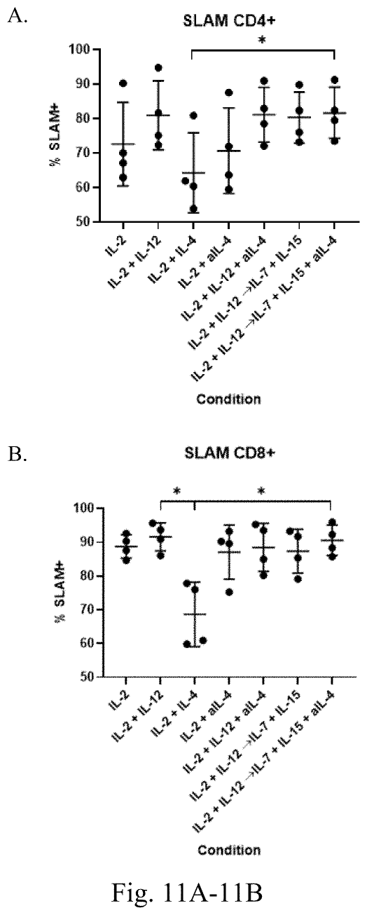

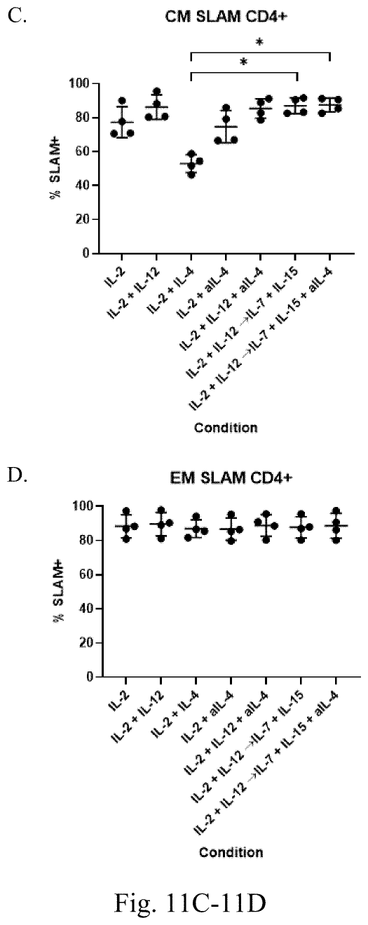

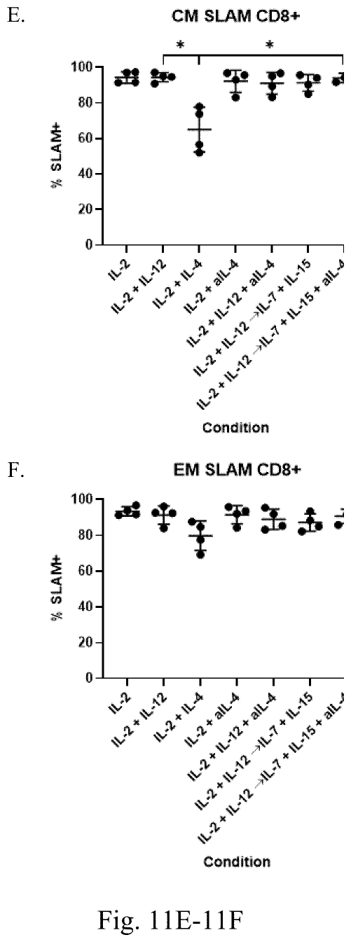

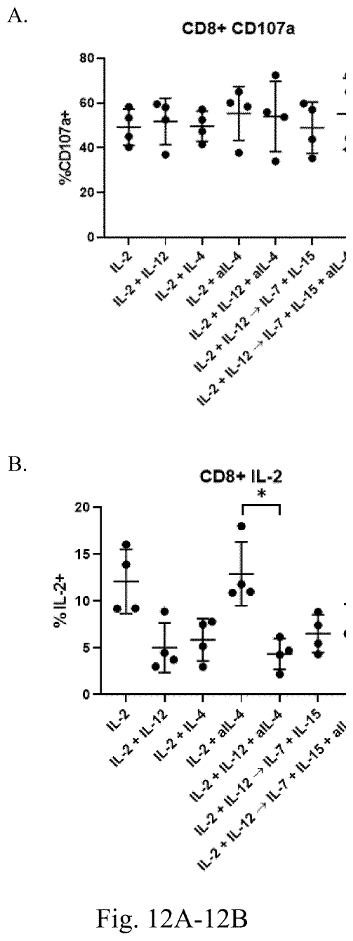

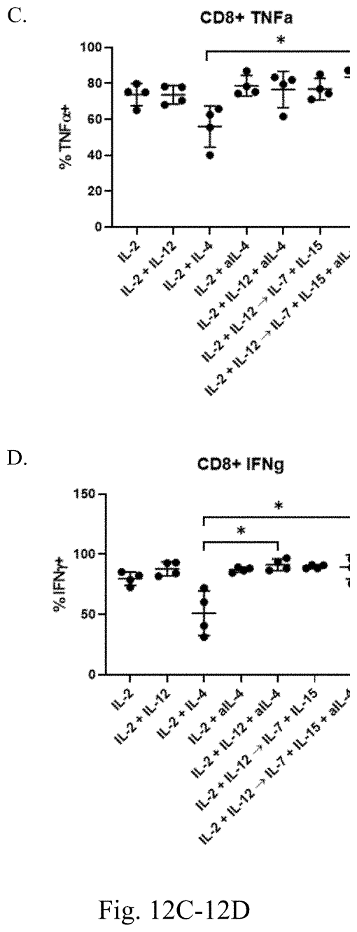

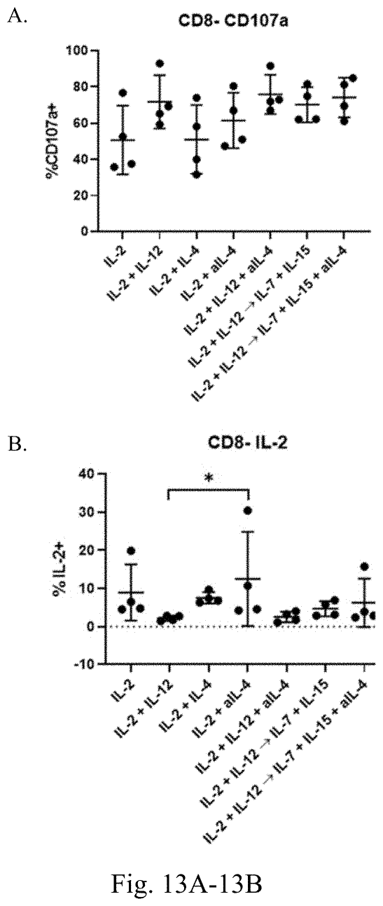

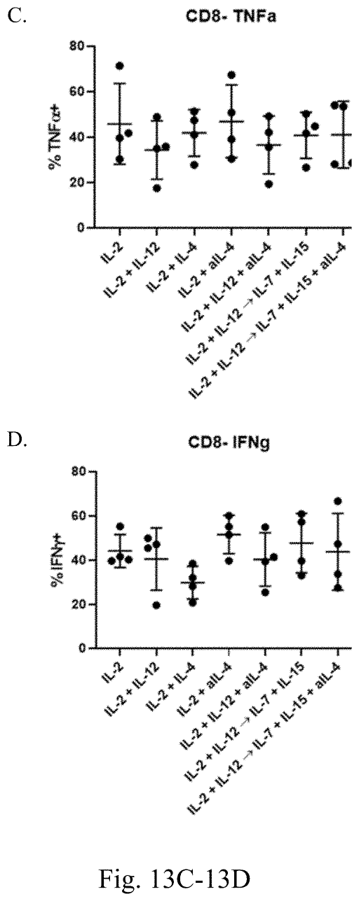

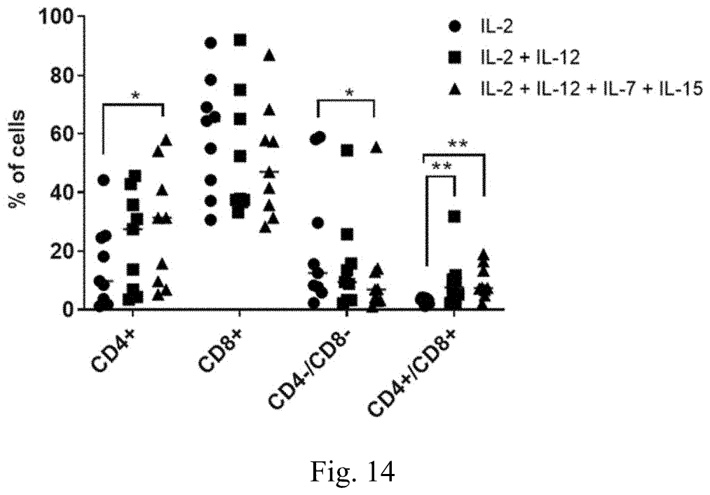

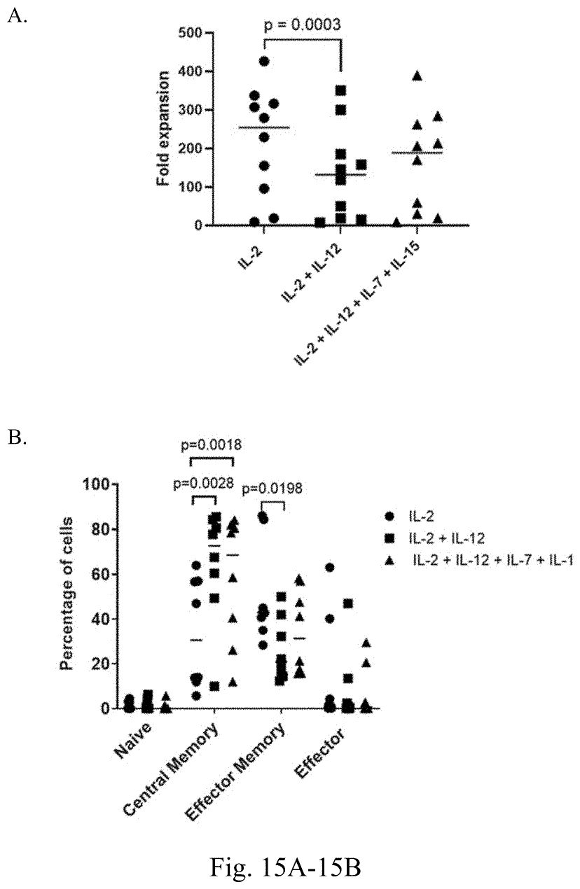

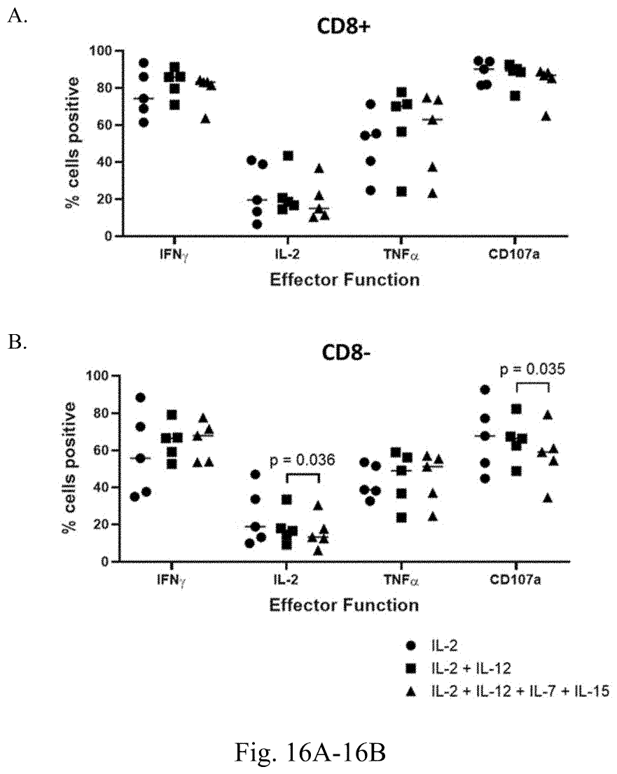

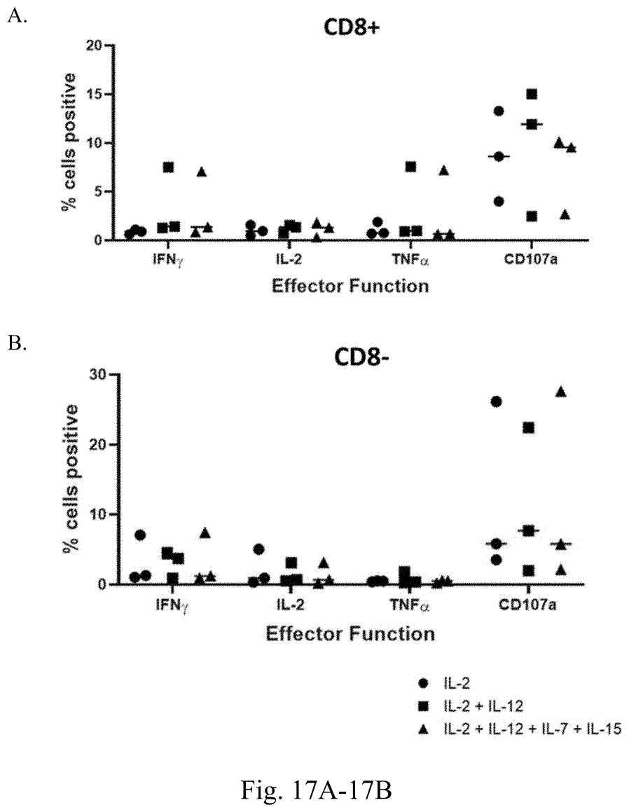

FIG. 3B—Expression of SLAM/CD150 in TIL pre- and post-rapid expansion protocol (REP). Post-REP TIL were stained with the following:

Fixable Viability Dye eFluor 450, αCD45RO FITC, αCD8 PE Vio770, αCD4 APC Cy7, αCD62L APC; and then counterstained with pairs of either mIgG1 PE and mIgG1 eFluor 710 isotype controls or αSLAM PE and αGITR eFluor 710. Cells were acquired on a MACSQuant analyser. Analysis was performed using MACSQuantify software. The proportion of SLAM+ cells were determined in each CD4+ or CD8+ population and plotted using Graphpad software. (A-C) SLAM expression in Pre-REP and Post-REP TIL from all melanoma subtypes; A) SLAM expression in all CD4+ and CD8+ T-cells; B) SLAM expression in naïve [N], memory [M] and effector [E] CD4+ TIL; C) SLAM expression in naïve [N], memory [M] and effector [E] CD8+ TIL; (D-F) SLAM expression in CD4+ and CD8+ TIL from cutaneous melanoma patients stratified by clinical response; D) SLAM expression on CD4+ and CD8+ TIL; E) SLAM expression in CD4+ T-cell subsets and F) SLAM expression in CD8+ subsets. Closed circles=progressive disease, open triangles=stable disease, open squares=responders. Response rates indicated are the best response achieved by the patient as determined by response evaluation criteria in solid tumours (RECISTv 1.1) measurements (refer to Schwartz et al. Eur J Cancer 2016) * P<0.05.

FIG. 4A-4B—Kaplan-Meier survival curve of patients correlating to SLAM expression—overall survival times of patients is plotted with two groups: in a first graph (A) SLAM high treated (patients with greater than 25% SLAM positive CD4 T-cells) and SLAM low treated (patients with less than 25% SLAM positive T-cells); and in a second graph (B) SLAM high treated (patients with greater than 40% SLAM positive CD4 T-cells) and SLAM low treated (patients with less than 40% SLAM positive T-cells).

FIG. 5A-5B—Viability and cytokine response in SLAM sorted cells—TIL from two donor final products, TIL032 and TIL054, were flow sorted for a SLAM High and SLAM low population. 24 h after culture viability was assessed (A) and the cells mixed with their respective matched autologous tumour cell line and cytokine response measured using flow cytometry (B).

FIG. 6A-6B—SLAM siRNA—SLAMF1 was measured by qPCR (A) or flow cytometry (B) in Raji, colorectal TIL (MRIBBO11) and melanoma TIL (TIL032) following 76 h treatment with SLAMF1 siRNA (siRNA) or untreated cells (control).

FIG. 7A-7B—SLAM overexpression—A SLAM expression cassette was created by cloning the human SLAMF1 sequence, 2A cleavage sequence and human cytoplasmic domain truncated CD19 sequence downstream of an EF1α promoter (A). Jurkat JRT3-T3.5 cells were transduced with titrating concentrations of lentiviral particles containing the SLAMF1 and truncated CD19 genes and expression analysed by flow cytometry (B).

FIG. 8A-8E—Effect of cytokine conditioning during rapid expansion protocol on TIL phenotype—TIL from four separate donors were expanded with mixed irradiated buffy coat feeders for 14 days under the indicated cytokine conditions before TIL counts were performed and CD4+, CD8+, central memory and effector memory phenotype was determined using flow cytometry.

FIG. 9A-9F—Effect of cytokine conditioning during rapid expansion protocol on SLAM expression—TIL from four separate donors were expanded with mixed irradiated buffy coat feeders for 14 days under the indicated cytokine conditions before SLAM expression was determined using flow cytometry.

FIG. 10A-10E—Effect of refined cytokine conditioning during rapid expansion protocol on TIL phenotype—TIL from four separate donors were expanded with mixed irradiated buffy coat feeders for 14 days under the indicated cytokine conditions before TIL counts were performed and CD4+, CD8+, central memory and effector memory phenotype was determined using flow cytometry.

FIG. 11A-11F—Effect of refined cytokine conditioning during rapid expansion protocol on SLAM expression—TIL from four separate donors were expanded with mixed irradiated buffy coat feeders for 14 days under the indicated cytokine conditions before SLAM expression was determined using flow cytometry.

FIG. 12A-12D—Effect of refined cytokine conditioning during rapid expansion protocol on CD8+ TIL effector activity—TIL from four separate donors were expanded with mixed irradiated buffy coat feeders for 14 days under the indicated cytokine conditions before the TIL were stimulated with K562 expressing a membrane bound OKT3 molecule and effector activity (CD107a, TNFα, IFNγ and IL-2) quantified by flow cytometry in the CD8+ population.

FIG. 13A-13D—Effect of refined cytokine conditioning during rapid expansion protocol on CD8− TIL effector activity—TIL from four separate donors were expanded with mixed irradiated buffy coat feeders for 14 days under the indicated cytokine conditions before the TIL were stimulated with K562 expressing a membrane bound OKT3 molecule and effector activity (CD107a, TNFα, IFNγ and IL-2) quantified by flow cytometry in the CD8− population.

FIG. 14—Effect of cytokine selection during outgrowth and REP on proportions of cells that are CD4+, CD8+, CD4-/CD8-, or CD4+/CD8+. TIL from nine donors were expanded in 3000 IU/ml IL-2 (●), IL-2 and an initial 25 ng/ml IL-12 (▪), or IL-2 and initial IL-12 followed by a switch to 10 ng/ml IL-7 and 10 ng/ml IL-15 (▴).

FIG. 15A-15B—Effect of cytokine selection during outgrowth and REP on proportions of cells that are phenotyped as central memory (CD45RO+/CD62L+), effector memory (CD45RO+/CD62L−), or effector cells. TIL from nine donors were expanded in 3000 IU/ml IL-2 (●), IL-2 and an initial 25 ng/ml IL-12 (▪), or IL-2 and initial IL-12 followed by a switch to 10 ng/ml IL-7 and 10 ng/ml IL-15 (▴).

FIG. 16A-16B—Effect of cytokine selection during outgrowth and REP on TILs cocultured with OKT3 expressing K562 cells. TILs from five donors were expanded in 3000 IU/ml IL-2 (●), IL-2 and an initial 25 ng/ml IL-12 (▪), or IL-2 and initial IL-12 followed by a switch to 10 ng/ml IL-7 and 10 ng/ml IL-15 (▴). Expanded TILs were cocultured with OKT3 expressing K562 cells and assessed for production of IFNγ, TNFα, IL-2 or CD107a within the (A) CD8+ and (B) CD8− cell populations.

FIG. 17A-17B—Effect of cytokine selection during outgrowth and REP on TILs cocultured with autologous tumour cell lines. TTLs from three donors were expanded in 3000 IU/ml IL-2 (●), IL-2 and an initial 25 ng/ml IL-12 (▪), or IL-2 and initial IL-12 followed by a switch to 10 ng/ml IL-7 and 10 ng/ml IL-15 (▴). Expanded TTLs were cocultured with matched autologous tumour cell lines and assessed for production of IFNγ, TNFα, IL-2 or CD107a within the (A) CD8+ and (B) CD8− cell populations.

### DETAILED DESCRIPTION OF THE INVENTION

Applicants and inventors hereon acknowledge, agree and reference international application Serial No. PCT/GB2019/050188, filed 23 Jan. 2019 by Immetacyte Limited and naming Nicola Kaye Price and John Stephen Bridgeman as inventors (“the '188 application”). The '188 application is prior filed but not published as to the present application and for purposes outside of the United States, the inventions herein meet the requirements of patentability as to this status of the '188 application. With regard to the United States, 35 USC § 102(b)(1) provides that a disclosure made one year or less before the effective filing date of a claimed invention shall not be prior art under 35 USC § 102(a)(1) with respect to the claimed invention if the disclosure was made by the inventor or joint inventor, and 35 USC § 102(b)(2) provides that a disclosure shall not be prior art under 35 USC § 102(a)(2) with respect to a claimed invention if the subject matter was disclosed or obtained directly or indirectly from the inventor or a joint inventor. 35 USC § 102(b)(2)(C) provides that a disclosure made in a US patent, US patent application publication, or WIPO published application shall not be prior art to a claimed invention under 35 USC § 102(a)(2) if, not later than the effective filing date of the claimed invention, the subject matter disclosed and the claimed invention were owned by the same person or subject to an obligation of assignment to the same person. Applicants and inventors hereon hereby provide a statement or attribution pursuant to inter alia 37 CFR § 1.130 and hereby acknowledge, agree, announce and declare under penalty of perjury under the laws of the US that they are familiar with the contents of the '188 application and the present application, and that the '188 application is disqualified under US law from being available as prior art, including because Nicola Kaye Price and John Stephen Bridgeman named hereon as inventors are inventors on the '188 application, i.e., the disclosure in the '188 application is made by an inventor or joint inventor hereon. Also, the '188 application and the present application include common ownership as Immetacyte Limited owns the '188 application and is an owner of the present application.

Immune cells, e.g. tumour reactive e.g. T cells for use in the method of the invention may be obtained using any method known in the art. In one embodiment, TILs or T cells that have infiltrated a tumor are isolated. TILs or T cells may be removed during surgery. TILs or T cells may be isolated after removal of tumor tissue by biopsy. TILs or T cells may be isolated by any means known in the art. In one embodiment the method may comprise obtaining a bulk population of TILs or T cells from a tumor sample by any suitable method known in the art. For example, a bulk population of TILs or T cells can be obtained from a tumor sample by dissociating the tumor sample into a cell suspension from which specific cell populations can be selected. Suitable methods of obtaining a bulk population of TILs or T cells may include, but are not limited to, any one or more of mechanically dissociating (e.g., mincing) the tumor, enzymatically dissociating (e.g., digesting) the tumor, and aspiration (e.g., as with a needle).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human. The tumor sample may be from any type of cancer as explained herein. In one embodiment, the tumor is ovarian cancer, lung cancer or melanoma.

Tumour reactive T cells can be obtained from a number of sources, including peripheral blood mononuclear cells (PBMC), bone marrow, lymph node tissue, spleen tissue, and tumors. In certain embodiments of the present invention, cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS), in an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, tumour reactive T cells are isolated from peripheral blood lymphocytes by lysing the red blood cells and depleting the monocytes, for example, by centrifugation through a PERCOLL™ gradient. A specific subpopulation of T cells, such as CD28⁺, CD4⁺, CDC, CD45RA⁺, and CD45RO⁺ T cells, can be further isolated by positive or negative selection techniques. For example, in one preferred embodiment, T cells are isolated by incubation with anti-CD3/anti-CD28 (i.e., 3.times.28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8⁺ T cells. The T cells can be cryopreserved and stored for later use. An acceptable duration of storage may be determined and validated and can be up to 6 months, up to a year, or longer.

In another embodiment, tumor infiltrating cells (TILs) are isolated and/or expanded from a tumor, for example by a fragmented, dissected, or enzyme digested tumor biopsy or mass. The TILs may be produced in a two-stage process using a tumor biopsy as the starting material: Stage 1 (generally performed over 2-3 hours) initial collection and processing of tumor material using dissection, enzymatic digestion and homogenization to produce a single cell suspension which can be directly cryopreserved to stabilize the starting material for subsequent manufacture and Stage 2 which can occur days or years later. Stage 2 may be performed over 4 weeks, which may be a continuous process starting with thawing of the product of Stage 1 and growth of the TIL out of the tumor starting material (about 2 weeks) followed by a rapid expansion process of the TIL cells (about 2 weeks) to increase the amount of cells and therefore dose. The TILs maybe concentrated and washed prior to formulation as a liquid suspension of cells. An exemplary TIL preparation is described in Applicant's U.S. patent application Ser. No. 62/951,559, filed Dec. 20, 2019.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14 cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™ In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, DYNAL® Magnetic Particle Concentrator (DYNAL MPC®). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific ‘I’ cells can be used. In certain embodiments, antigen-specific T cells can be isolated from a patient of interest, such as a patient afflicted with a cancer or an infectious disease. In one embodiment neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation And Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-WIC tetramers (Altman, et al., Science. 1996 Oct. 4; 274(5284):94-6). In another embodiment the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-WIC tetramers can be generated using techniques known in the art and can be made with any WIC molecule of interest and any antigen of interest as described herein. Specific epitopes to be used in this context can be identified using numerous assays known in the art. For example, the ability of a polypeptide to bind to WIC class I may be evaluated indirectly by monitoring the ability to promote incorporation of ¹²⁵I labeled β2-microglobulin (32m) into WIC class I/β2m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting the T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.) FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of TILs or T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

The present invention relates to T-cells, for example T-cells present in a sample of Tumour-infiltrating lymphocytes (TIL). In particular, it relates to cell populations comprising T-cells such as populations of TTLs which are enriched for tumour-reactive T-cells.

Tumour-infiltrating lymphocytes are white blood cells that have left the bloodstream and migrated into a tumour. They are mononuclear immune cells, a mix of different types of cells (i.e., T-cells, B-cells, NK cells, macrophages) in variable proportions, T-cells being by far the most abundant cells. They can often be found in the stroma and within the tumour itself.

TTLs may specifically recognize, lyse, and/or kill tumour cells. The presence of lymphocytes in tumours is often associated with better clinical outcomes. Thus, TILs can be functionally defined by their ability to infiltrate solid tumors upon reintroduction into a patient. TTLs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25.

T-cells or T-lymphocytes are a type of lymphocyte that has a central role in cell-mediated immunity. They can be distinguished from other lymphocytes, such as B-cells and natural killer cells (NK cells), by the presence of a T-cell receptor (TCR) on the cell surface. There are various types of T-cell, as summarised below.

Cytolytic T-cells (TC cells, or CTLs) destroy virally infected cells and tumour cells, and are also implicated in transplant rejection. CTLs express the CD8 molecule at their surface. These cells recognize their targets by binding to antigen associated with MHC class I, which is present on the surface of all nucleated cells. Through IL-10, adenosine and other molecules secreted by regulatory T-cells, the CD8+ cells can be inactivated to an anergic state, which prevents autoimmune diseases such as experimental autoimmune encephalomyelitis.

Memory T-5 cells are a subset of antigen-specific T-cells that persist long-term after an infection has resolved. They quickly expand to large numbers of effector T-cells upon re-exposure to their cognate antigen, thus providing the immune system with “memory” against past infections. Memory T-cells comprise three subtypes: central memory T-cells (TCM cells) and two types of effector memory T-cells (TEM cells and TEMRA cells). Memory cells may be either CD4+ or CD8+. Memory T-cells typically express the cell surface protein CD45RO.

Regulatory T-cells (Treg cells), formerly known as suppressor T-cells, are crucial for the maintenance of immunological tolerance. Their major role is to shut down T-cell-mediated immunity toward the end of an immune reaction and to suppress auto-reactive T-cells that escaped the process of negative selection in the thymus.

CD4+ T cells are commonly divided into regulatory T (Treg) cells and conventional T helper (Th) cells. Two major classes of CD4+ Treg cells have been described naturally occurring Treg cells and adaptive Treg cells.

Naturally occurring Treg cells (also known as CD4+CD25+FoxP3+ Treg cells) arise in the thymus and have been linked to interactions between developing T-cells with both myeloid (CD11c+) and plasmacytoid (CD123+) dendritic cells that have been activated with TSLP. Naturally occurring Treg cells can be distinguished from other T-cells by the presence of an intracellular molecule called FoxP3.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B- and T-lymphocytes.

As used herein, the term “modulation of at least one function of the immune cell” includes the modulation of any of a variety of T cell-related functions and/or activities, including by way of non-limiting example, controlling or otherwise influencing the networks that regulate T cell differentiation; controlling or otherwise influencing the networks that regulate T cell maintenance, for example, over the lifespan of a T cell; controlling or otherwise influencing the networks that regulate T cell function; controlling or otherwise influencing the networks that regulate helper T cell (Th cell) differentiation; controlling or otherwise influencing the networks that regulate Th cell maintenance, for example, over the lifespan of a Th cell; controlling or otherwise influencing the networks that regulate Th cell function; controlling or otherwise influencing the networks that regulate Th17 cell differentiation; controlling or otherwise influencing the networks that regulate Th17 cell maintenance, for example, over the lifespan of a Th17 cell; controlling or otherwise influencing the networks that regulate Th17 cell function; controlling or otherwise influencing the networks that regulate regulatory T cell (Treg) differentiation; controlling or otherwise influencing the networks that regulate Treg cell maintenance, for example, over the lifespan of a Treg cell; controlling or otherwise influencing the networks that regulate Treg cell function; controlling or otherwise influencing the networks that regulate other CD4⁺ T cell differentiation; controlling or otherwise influencing the networks that regulate other CD4⁺ T cell maintenance; controlling or otherwise influencing the networks that regulate other CD4⁺ T cell function; controlling or otherwise influencing the networks that regulate other CD8⁺ T cell differentiation; controlling or otherwise influencing the networks that regulate other CD8T cell maintenance; or controlling or otherwise influencing the networks that regulate other CD8⁺ T cell function.

The term “isolated” with reference to a particular component generally denotes that such component exists in separation from—for example, has been separated from or prepared and/or maintained in separation from—one or more other components of its natural environment. More particularly, the term “isolated” as used herein in relation to a cell or cell population denotes that such cell or cell population does not form part of an animal or human body.

The term “immune cell” as used herein generally encompasses any cell derived from a hematopoietic stem cell that plays a role in the immune response. Immune cells include, without limitation, lymphocytes, such as T cells and B cells, antigen-presenting cells (APC), dendritic cells, monocytes, macrophages, natural killer (NK) cells, mast cells, basophils, eosinophils, or neutrophils, as well as any progenitors of such cells. In certain preferred embodiments, the immune cell may be a T cell. As used herein, the term “T cell” (i.e., T lymphocyte) is intended to include all cells within the T cell lineage, including thymocytes, immature T cells, mature T cells and the like. The term “T cell” may include CD4⁺ and/or CD8⁺ T cells, T helper (Tₕ) cells, e.g., Tₕ₁, Tₕ₂ and Tₕ₁₇ cells, and T regulatory (T_{reg}) cells.

In certain more preferred embodiments, the immune cell is a CD8⁺ T cell, also known as cytotoxic T cell or Tc. A CD8⁺ T cell is a T cell expressing the CD8 cell surface marker, and recognizes antigens in the context of MHC class I presentation. CD8⁺ T cells have cytotoxic activity and proliferate in response to IFN-gamma and other cytokines. Engagement of CD8⁺ T-cell to the TCR receptor of a CD8⁺ T-cell antigen presented by Class I MHC molecules and co-stimulating molecules lead to cytotoxic activity, proliferation and/or cytokine production. In other embodiments, the immune cell is a CD4⁺ T cell (i.e. a CD4+ T helper cell).

The term “modified” as used herein broadly denotes that an immune cell has been subjected to or manipulated by a man-made process, such as a man-made molecular- or cell biology process, resulting in the modification of at least one characteristic of the immune cell. Such man-made process may for example be performed in vitro or ex vivo.

The term “altered expression” denotes that the modification of the immune cell alters, i.e., changes or modulates, the expression of the recited gene(s) or polypeptides(s). The term “altered expression” encompasses any direction and any extent of said alteration. Hence, “altered expression” may reflect qualitative and/or quantitative change(s) of expression, and specifically encompasses both increase (e.g., activation or stimulation) or decrease (e.g., inhibition) of expression.

The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e., absent level as compared to a reference sample), or any decrease between 10-100% as compared to a reference level, as that term is defined herein. The term “abolish” or “abolished” may in particular refer to a decrease by 100%, i.e., absent level as compared to a reference sample.

The modification may produce an immune cell comprising altered expression or activity of CD150/SLAM/SLAMF1 or of the one or more genes or gene products as taught herein; or the modification may produce an immune cell which does not comprise altered expression or activity of CD150/SLAM/SLAMF1 or of the one or more genes or gene products as taught herein, but which has acquired the ability to exhibit altered expression or activity of CD150/SLAM/SLAMF1 or of the one or more genes or gene products as taught herein in response to an external signal. The latter cell has thus been modified to comprise an agent capable of inducibly (i.e., in response to a signal, more particularly to an external signal, such as to an external chemical, biological and/or physical signal) altering expression or activity of CD150/SLAM/SLAMF1 or of the one or more genes or gene products as taught herein.

Hence, in certain embodiments, the modification may comprise exposing the immune cell to an agent or contacting the immune cell with an agent or introducing into the immune cell an agent capable of altering the expression or activity of CD150/SLAM/SLAMF1 or of the one or more genes or gene products as taught herein, whereby the expression or activity of CD150/SLAM/SLAMF1 or of the one or more genes or gene products as taught herein in the immune cell is altered. In certain embodiments, the agent or one or more elements thereof may be under inducible control. For example, the expression of the agent or one or more elements thereof by the immune cell and/or the activity of the agent or one or more elements thereof in the cell may be under inducible control. The immune cell thereby acquires the ability to exhibit altered expression or activity of CD150/SLAM/SLAMF1 or of the one or more genes or gene products as taught herein in response to an external signal configured to modulate the agent or one or more elements thereof, such as the expression and/or the activity of the agent or one or more elements thereof.

Any one or more of the several successive molecular mechanisms involved in the expression of a given gene or polypeptide may be targeted by the immune cell modification as intended herein. Without limitation, these may include targeting the gene sequence (e.g., targeting the polypeptide-encoding, non-coding and/or regulatory portions of the gene sequence), the transcription of the gene into RNA, the polyadenylation and where applicable splicing and/or other post-transcriptional modifications of the RNA into mRNA, the localisation of the mRNA into cell cytoplasm, where applicable other post-transcriptional modifications of the mRNA, the translation of the mRNA into a polypeptide chain, where applicable post-translational modifications of the polypeptide, and/or folding of the polypeptide chain into the mature conformation of the polypeptide. For compartmentalised polypeptides, such as secreted polypeptides and transmembrane polypeptides, this may further include targeting trafficking of the polypeptides, i.e., the cellular mechanism by which polypeptides are transported to the appropriate sub-cellular compartment or organelle, membrane, e.g. the plasma membrane, or outside the cell.

Hence, “altered expression” may particularly denote altered production of the recited gene products by the modified immune cell. As used herein, the term “gene product(s)” includes RNA transcribed from a gene (e.g., mRNA), or a polypeptide encoded by a gene or translated from RNA.

Also, “altered expression” as intended herein may encompass modulating the activity of CD150/SLAM/SLAMF1 and/or of the one or more genes or gene products as taught herein. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein, “modulating” or “to modulate” generally means either affecting the activity of a target or antigen, e.g., CD150/SLAM/SLAMF1 and/or the one or more genes or gene products as taught herein, and advantageously increasing the activity of the target or antigen, e.g., CD150/SLAM/SLAMF1 and/or the one or more genes or gene products as taught herein, as measured using a suitable in vitro, cellular or in vivo assay. In particular, “modulating” or “to modulate” generally relates to increasing the (relevant or intended) biological activity of the target or antigen, e.g., CD150/SLAM/SLAMF1 and/or the one or more genes or gene products as taught herein, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, e.g., CD150/SLAM/SLAMF1 and/or the one or more genes or gene products as taught herein, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

The term “agent” as used herein generally refers to any substance or composition, such as a chemical entity or biological product, or combination of chemical entities or biological products, capable of achieving a desired effect in a system, more particularly in a biological system, e.g., in a cell, tissue, organ, or an organism. In the present context, an agent may be exposed to, contacted with or introduced into an immune cell to modify at least one characteristic of the immune cell, such as to (inducibly) alter the expression or activity of CD150/SLAM/SLAMF1 or of the one or more genes or gene products as taught herein by the immune cell. Further in the present context, an agent may be administered to a subject to treat or prevent or control a disease or condition, for example by (inducibly) altering the expression or activity of CD150/SLAM/SLAMF1 or of the one or more genes or gene products as taught herein by immune cells of the subject.

The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of downstream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g., an adenine, “A”, a guanine, “G”, a thymine “T”, or a cytosine, “C”) or RNA (e.g., an A, a G, an uracil, “U”, or a C). The term “nucleic acid” encompasses the terms “oligonucleotide” and “polynucleotide” each as a subgenus of the term “nucleic acid”. The term “oligonucleotide” refers to a molecule of between about 3 and about 100 nucleobases in length. The term “polynucleotide” refers to at least one molecule of greater than about 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, have a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides can exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. In some embodiments, a CD150/SLAM/SLAMF1 modulator or a modulator of any one of the one or more gene products as taught herein comprises a CD150/SLAM/SLAMF1 protein or fragment thereof, or comprises the gene product or fragment thereof, respectively, fused to a Fc fragment, which is comprised of D- or L-amino acid residues, as use of naturally occurring L-amino acid residues has the advantage that any break-down products should be relatively non-toxic to the cell or organism.

In yet a further embodiment, a CD150/SLAM/SLAMF1 modulator, or a modulator of the one or more gene products as taught herein, which is a peptide or fragments or derivatives thereof can be a retro-inverso peptide. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous a-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP0097994B) which is also incorporated herein in its entirety by reference.

In some embodiments, as further explained herein, the modulator is a cytokine, in particular one that is a Type 1 T helper (Th1) skewing cytokine as further defined herein.

The term “antibody” is meant to be an immunoglobulin protein that is capable of binding an antigen. Antibody as used herein is meant to include antibody fragments, e.g. F(ab′)2, Fab′, Fab, capable of binding the antigen or antigenic fragment of interest. Exemplary fragments include Fab, Fab′, F(ab′)2, Fabc, Fd, single domain antibody, e.g. a VHH, humanized VHH, VH, camelised VH or VL, heavy chain only antibody and scFv and/or Fv fragments. As used herein, the term “antibody” is used in its broadest sense and generally refers to any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The term antibody thereby includes a whole immunoglobulin molecule, a monoclonal antibody, a chimeric antibody, a humanized antibody, a human antibody, or an immunologically effective fragment of any of these. The term thus specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

The term “humanized antibody” is used herein to describe complete antibody molecules, i.e. composed of two complete light chains and two complete heavy chains, as well as antibodies consisting only of antibody fragments, e.g. Fab, Fab′, F(ab′)₂, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human subject.

All gene name symbols refer to the gene as commonly known in the art. Gene symbols may be those referred to by the HUGO Gene Nomenclature Committee (HGNC). Any reference to the gene symbol is a reference made to the entire gene or variants of the gene. The HUGO Gene Nomenclature Committee is responsible for providing human gene naming guidelines and approving new, unique human gene names and symbols. All human gene names and symbols can be searched at www.genenames.org, the HGNC website, and the guidelines for their formation are available there (www.genenarnes.org/guidelines). Hence, the gene symbols as used throughout this specification may particularly preferably refer to the respective human genes.

In one aspect, the present invention provided herein relates to obtaining and using populations of T-cells, including T-cells present in tumour-infiltrating lymphocytes (TILs) populations, that are enriched for tumour reactive T-cells by selecting T-cells expressing CD150/SLAM/SLAMF1 from the bulk population of cells. When selected, the CD150/SLAM/SLAMF1 positive cells are separated from the bulk population to provide a population enriched for T-cells expressing CD150/SLAM/SLAMF1, and optionally the selected cells are then expanded to increase the number of CD150/SLAM/SLAMF1 +ve (positive) cells. Once CD150/SLAM/SLAMF1 +ve cells have been selected for, any suitable T-cell expansion method known in the art can be used, providing CD150/SLAM/SLAMF1 expression is maintained post expansion.

For example, described herein is an in vitro method of selecting cells expressing prognostically-favourable levels of said biological marker (CD150/SLAM/SLAMF1) using one or more of the following selection techniques: (i) flow cytometry, (ii) antibody panning, (iii) magnetic selection, (iv) biomarker targeted cell enrichment.

Like many other human genes, there are multiple isoforms of SLAMF1, some of which are noncoding or do not generate a functional surface protein. However, these splice variants differ in their cytoplasmic domain, and antibodies to SLAMF1, such as those described herein, bind the extracellular domain and thus bind the different isoforms of SLAMF1.

Selected cells expressing the said biological marker (CD150/SLAM/SLAMF1) may then, for example, be expanded by way of one or both of the following options:

a) Irradiated feeder cells in such a fashion as to provide a T-cell activation signal and costimulation driven by antibodies or costimulatory receptors; and/or

b) Immobilised or soluble reagents which provide a T-cell activation signal and costimulation signals driven through said one or more biological markers.

For example, in some embodiments, costimulation through SLAM/CD150 using antibodies may be feasible as they have been shown to induce costimulation in the literature, see: Aversa G. Chang C C, Carballido J M, Cocks B G, de Vries J E. J Immunol. 1997 May 1; 158(9):4036-44.

In one aspect, the present invention provides a cell which comprises an exogenous nucleic acid molecule encoding SLAM (Signaling lymphocyte activation molecule), also termed /SLAMF1/CD150/1/CD150/CDw150/IPO-3; Human amino acid sequence, see: NCBI Reference Sequence: NP_003028.1.

The word “exogenous” means that the nucleic acid molecule is made by recombinant means and is introduced into the cell e.g. by way of a vector, such as a lentiviral vector. The cell is engineered to contain the nucleic acid molecule and to express (or over-express) SLAM/SLAMF1/CD150. Optionally, the cell may further comprise a second exogenous nucleic acid for example encoding a Chimeric Antigen Receptor (CAR) or a T-cell receptor (TCR) and so also express a CAR or TCR.

As used herein, the terms “polynucleotide”, “nucleotide”, and “nucleic acid” are intended to be synonymous with each other.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed, e.g. codon optimisation.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides may be modified by any method available in the art. Such modifications may be carried out in order to enhance the in vivo activity or life span of the polynucleotides of interest.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

In one embodiment, any of the targets described herein are modulated in CAR T cells before administering to a patient in need thereof, preferably, CD150/SLAM/SLAMF1. Not being bound by a theory, modulating the expression or activity of a gene related to dysfunction increases the activity of the T cell. Not being bound by a theory, modulating the expression or activity of a gene related to activation increases the activity of the T cell.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. These definitions generally refer to a single-stranded molecule, but in specific embodiments will also encompass an additional strand that is partially, substantially or fully complementary to the single-stranded molecule. Thus, a nucleic acid may encompass a single-stranded molecule or a double-stranded molecule that comprises one or more complementary strand(s) or “complement(s)” of a particular sequence comprising a molecule. As used herein, a single-stranded nucleic acid may be denoted by the prefix “ss”, a double stranded nucleic acid by the prefix “ds”, and a triple stranded nucleic acid by the prefix “is”. The term “gene” may refer to the segment of DNA involved in producing a polypeptide chain, it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto.

A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

Hence, the endogenous target gene, e.g., CD150/SLAM/SLAMF1 gene may be modified or “mutated”. Any types of mutations achieving the intended effects are contemplated herein. For example, suitable mutations may include deletions, insertions, and/or substitutions, The term “deletion” refers to a mutation wherein one or more nucleotides, typically consecutive nucleotides, of a nucleic acid are removed, i.e., deleted, from the nucleic acid. The term “insertion” refers to a mutation wherein one or more nucleotides, typically consecutive nucleotides, are added, i.e., inserted, into a nucleic acid. The term “substitution” refers to a mutation wherein one or more nucleotides of a nucleic acid are each independently replaced, i.e., substituted, by another nucleotide.

In certain other embodiments, a mutation may be a substitution of one or more nucleotides in the ORF encoding the target protein, e.g., CD150/SLAM/SLAMF1 resulting in substitution of one or more amino acids of the target protein, e.g., CD150/SLAM/SLAMF1. Such mutation may typically preserve the production of the polypeptide, and may preferably affect, such as diminish or abolish, some or all biological function(s) of the polypeptide. The skilled person can readily introduce such substitutions.

In certain preferred embodiments, a mutation may abolish native splicing of a pre-mRNA encoding the target protein, e.g., CD150/SLAM/SLAMF1. In the absence of native splicing, the pre-mRNA may be degraded, or the pre-mRNA may be alternatively spliced, or the pre-mRNA may be spliced improperly employing latent splice site(s) if available. Hence, such mutation may typically effectively abolish the production of the polypeptide's mRNA and thus the production of the polypeptide. Various ways of interfering with proper splicing are available to a skilled person, such as for example but without limitation, mutations which alter the sequence of one or more sequence elements required for splicing to render them inoperable, or mutations which comprise or consist of a deletion of one or more sequence elements required for splicing. The terms “splicing”, “splicing of a gene”, “splicing of a pre-mRNA” and similar as used herein are synonymous and have their art-established meaning. By means of additional explanation, splicing denotes the process and means of removing intervening sequences (introns) from pre-mRNA in the process of producing mature mRNA. The reference to splicing particularly aims at native splicing such as occurs under normal physiological conditions. The terms “pre-mRNA” and “transcript” are used herein to denote RNA species that precede mature mRNA, such as in particular a primary RNA transcript and any partially processed forms thereof. Sequence elements required for splicing refer particularly to cis elements in the sequence of pre-mRNA which direct the cellular splicing machinery (spliceosome) towards correct and precise removal of introns from the pre-mRNA. Sequence elements involved in splicing are generally known per se and can be further determined by known techniques including inter alia mutation or deletion analysis. By means of further explanation, “splice donor site” or “5′ splice site” generally refer to a conserved sequence immediately adjacent to an exon-intron boundary at the 5′ end of an intron. Commonly, a splice donor site may contain a dinucleotide GU, and may involve a consensus sequence of about 8 bases at about positions +2 to −6. “Splice acceptor site” or “3′ splice site” generally refers to a conserved sequence immediately adjacent to an intron-exon boundary at the 3′ end of an intron. Commonly, a splice acceptor site may contain a dinucleotide AG, and may involve a consensus sequence of about 16 bases at about positions −14 to +2.

In certain embodiments, the endogenous target gene, e.g., endogenous CD150/SLAM/SLAMF1 gene may be modified using a nuclease.

The term “nuclease” as used herein broadly refers to an agent, for example a protein or a small molecule, capable of cleaving a phosphodiester bond connecting nucleotide residues in a nucleic acid molecule. In some embodiments, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some embodiments, a nuclease may recognize a single stranded target site, in other embodiments a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 5′ end of the respective DNA strand.

The nuclease may introduce one or more single-strand nicks and/or double-strand breaks in the endogenous target gene, e.g., endogenous CD150/SLAM/SLAMF1 gene, whereupon the sequence of the endogenous target gene, e.g., endogenous CD150/SLAM/SLAMF1 gene may be modified or mutated via non-homologous end joining (NHEJ) or homology-directed repair (HDR).

In certain embodiments, the nuclease may comprise (i) a DNA-binding portion configured to specifically bind to the endogenous target gene, e.g., endogenous CD150/SLAM/SLAMF1 gene and (ii) a DNA cleavage portion. Generally, the DNA cleavage portion will cleave the nucleic acid within or in the vicinity of the sequence to which the DNA-binding portion is configured to bind.

In certain embodiments, the DNA-binding portion may comprises a zinc finger protein or DNA-binding domain thereof, a transcription activator-like effector (TALE) protein or DNA-binding domain thereof, or an RNA-guided protein or DNA-binding domain thereof.

In certain embodiments, the DNA-binding portion may comprise (i) Cas9 or Cpf1 or any Cas protein described herein modified to eliminate its nuclease activity, or (ii) DNA-binding domain of Cas9 or Cpf1 or any Cas protein.

In certain embodiments, the DNA cleavage portion comprises FokI or variant thereof or DNA cleavage domain of Fold or variant thereof.

In certain embodiments, the nuclease may be an RNA-guided nuclease, such as Cas9 or Cpf1 or any Cas.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

In an aspect, the present invention provides a vector which comprises a nucleic acid sequence or nucleic acid construct of the invention.

Such a vector may be used to introduce the nucleic acid sequence(s) or nucleic acid construct(s) into a host cell so that it expresses SLAM/SLAMF1/CD150.

The vector may, for example, be a plasmid or a viral vector, such as a retroviral vector or a lentiviral vector, or a transposon based vector or synthetic mRNA. Vectors derived from retroviruses, such as the lentivirus, are suitable tools to achieve long-term gene transfer since they allow long-term, stable integration of a transgene or transgenes and its propagation in 30 daughter cells.

The vector may be capable of transfecting or transducing a T-lymphocyte. The present invention also provides vectors in which a nucleic acid of the present invention is inserted.

The expression of natural or synthetic nucleic acids encoding SLAM/SLAMF1/CD150, and optionally a TCR or CAR is typically achieved by operably linking a nucleic acid encoding the SLAM/SLAMF1/CD150 and TCR/CAR polypeptide or portions thereof to one or more promoters, and incorporating the construct into an expression vector. The vectors can be suitable for replication and integration in eukaryotic cells. Typical cloning vectors contain transcription and translation terminators, initiation sequences, and promoters useful for regulation of the expression of the desired nucleic acid sequence.

Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193).

In order to assess the expression of the SLAM/SLAMF1/CD150 polypeptide or portions thereof, the expression vector to be introduced into a cell can also contain either a selectable marker gene or a reporter gene or both to facilitate identification and selection of expressing cells from the population of cells sought to be transfected or transduced through viral vectors. For example, the marker gene may be CD19 as shown in FIG. 7A.

In some embodiments, the nucleic acid construct is as shown in FIG. 7A. FIG. 7A shows a lentiviral expression construct created in which SLAMF1 and a CD19 marker gene are driven by an EF1α promoter.

The present invention also relates to a pharmaceutical composition containing a population of cells, T-cells or TIL(s) of the invention together with a pharmaceutically acceptable carrier, diluent or excipient, and optionally one or more further pharmaceutically active polypeptides and/or compounds. Such a formulation may, for example, be in a form suitable for intravenous infusion. Provided herein is a pharmaceutical composition for intravenous infusion comprising a population of T-cells wherein at least 25%, 30%, 40%, or 50% of the T-cells express SLAM/SLAMF1/CD150. In certain embodiments, the immune cell as intended herein, such as a T cell, preferably a CD8+ T cell, may display tumor specificity. By means of an example, the immune cell, such as a T cell, preferably a CD8+ T cell, may have been isolated from a tumor of a subject. More preferably, the immune cell may be a tumor infiltrating lymphocyte (TIL). Generally, “tumor infiltrating lymphocytes” or “TILs” refer to white blood cells that have left the bloodstream and migrated into a tumor. Such T cells typically endogenously express a T cell receptor having specificity to an antigen expressed by the tumor cells (tumor antigen specificity).

In alternative embodiments, an immune cell, such as a T cell, preferably a CD8⁺ T cell, may be engineered to express a T cell receptor having specificity to a desired antigen, such as a tumor cell antigen. For example, the immune cell, such as a T cell, preferably a CD8⁺ T cell, may comprise a chimeric antigen receptor (CAR) having specificity to a desired antigen, such as a tumor-specific chimeric antigen receptor (CAR).

A “pharmaceutical composition” refers to a composition that usually contains an excipient, such as a pharmaceutically acceptable carrier that is conventional in the art and that is suitable for administration to cells or to a subject. In addition, compositions for topical (e.g., oral mucosa, respiratory mucosa) and/or oral administration can be in the form of solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21 st Ed.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, with which a modulator as described herein is combined in a formulation to be administered to a subject. Each carrier must be “acceptable” in the sense of being compatible with the other ingredients of the formulation, as well as in the sense of not being toxic or provoking undue side effects in an individual. Pharmaceutically acceptable carriers are well known to those of skill in the art.

A further aspect provides the isolated immune cell or the cell population as taught herein for use in therapy.

A further aspect provides the isolated immune cell or the cell population as taught herein for use in immunotherapy or adoptive immunotherapy, preferably immunotherapy or adoptive immunotherapy of a proliferative disease, such as a tumor or cancer, or a chronic infection, such as a chronic viral infection. Certain embodiments provide the isolated immune cell or the cell population as taught herein for use in immunotherapy or adoptive immunotherapy in a subject, wherein the subject has been determined to comprise immune cells which: express CD150/SLAM/SLAMF1; are dysfunctional, or are not dysfunctional; or express a signature of dysfunction as described herein in this specification. Thus, one aspect provides an enriched and expanded cell population of tumour reactive T-cells for use in the treatment of cancer, comprising administering to a cancer patient in need thereof cells from a cell population, wherein the cell population is prepared by a method comprising identifying and/or obtaining a cell population expressing CD150/SLAM/SLAMF1 and expanding the cell population. Also provided is an enriched and expanded cell population of tumour reactive T-cells, wherein the cell population is prepared by a method comprising identifying and/or obtaining a cell population expressing CD150/SLAM/SLAMF1 and expanding the cell population for us in the manufacture of a medicament for the treatment of cancer.

The term “immunotherapy” broadly encompasses therapeutic or prophylactic treatments aimed at modulating, such as upregulating or downregulating, immune response in a subject.

As used herein, “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, indisposition, or affliction.

The term “proliferative disease or disorder” generally refers to any disease or disorder characterized by neoplastic cell growth and proliferation, whether benign, pre-malignant, or malignant. The term proliferative disease generally includes all transformed cells and tissues and all cancerous cells and tissues. Proliferative diseases or disorders include, but are not limited to abnormal cell growth, benign tumours, premalignant or precancerous lesions, malignant tumors, and cancer.

The terms “tumor” or “tumor tissue” refer to an abnormal mass of tissue resulting from excessive cell division. A tumor or tumor tissue comprises “tumor cells” which are neoplastic cells with abnormal growth properties and no useful bodily function. Tumors, tumor tissue and tumor cells may be benign, pre-malignant or malignant, or may represent a lesion without any cancerous potential. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

The term “cancer” refers to a malignant neoplasm characterized by deregulated or unregulated cell growth. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor. The term “metastatic” or “metastasis” generally refers to the spread of a cancer from one organ or tissue to another non-adjacent organ or tissue. The occurrence of the proliferative disease in the other non-adjacent organ or tissue is referred to as metastasis.

In certain embodiments, the proliferative disease may be selected from the group consisting of melanoma, lung cancer, squamous cell cancer, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, head cancer and neck cancer. In one embodiment, the cancer is selected from the group consisting of ovarian cancer, lung cancer and melanoma.

The disclosed methods may further be used in combination with other known cancer therapies. In some embodiments, the method may further comprise administering to the subject an anti-cancer agent. In some embodiments, the anti-cancer agent may comprise at least one of cisplatin, oxaliplatin, a kinase inhibitor, trastuzumab, cetuximab, panitumumab, lambrolizumab and nivolumab.

As used herein, the terms “treat,” “treating” and “treatment” refer to the alleviation or measurable lessening of one or more symptoms or measurable markers of a disease or disorder; while not intending to be limited to such, disease or disorders of particular interest include autoimmune diseases, chronic infection and cancer. Measurable lessening includes any statistically significant decline in a measurable marker or symptom. In some embodiments, treatment is prophylactic treatment.

The term “therapeutically effective amount” refers to an amount effective, at dosages and for periods of time necessary, to achieve the desired therapeutic result, e.g., a diminishment or prevention of effects associated with various disease states or conditions. The term “therapeutically effective amount” refers to an amount of a cell population, target gene or gene product modulator, e.g., a CD150/SLAM/SLAMF1 modulator as disclosed herein, advantageously agent that increases CD150/SLAM/SLAMF1 expression, effective to treat or prevent a disease or disorder in a mammal. A therapeutically effective amount of a target gene or gene product modulator, e.g., a CD150/SLAM/SLAMF1 modulator, advantageously agent that increases CD150/SLAM/SLAMF1 expression, can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an “effective amount”, which as used herein refers to the amount of therapeutic agent of pharmaceutical composition to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of an immune or autoimmune disease which are appropriate measures by those skilled in the art. It should be noted that a target gene or gene product modulator, e.g., CD150/SLAM/SLAMF1 modulator as disclosed herein can be administered as a pharmaceutically acceptable salt and can be administered alone or as an active ingredient in combination with pharmaceutically acceptable carriers, diluents, adjuvants and vehicles.

The term “prophylactically effective amount” refers to an amount of a cell population, target gene or gene product modulator, e.g., a CD150/SLAM/SLAMF1 modulator which is effective, at dosages and for periods of time necessary, to achieve the desired prophylactic result, e.g., the amount of a target gene or gene product modulator, e.g., a CD150/SLAM/SLAMF1 activator to reduce a symptom of a chronic immune disease, e.g., a chronic infection or to treat cancer in the subject. Typically, since a prophylactic dose of a target gene or gene product modulator, e.g., a CD150/SLAM/SLAMF1 modulator is administered to a subject prior to or at an earlier stage of a disease, and in some embodiments, a prophylactically effective amount is less than the therapeutically effective amount. A prophylactically effective amount of a target gene or gene product modulator, e.g., a CD150/SLAM/SLAMF1 modulator is also one in which any toxic or detrimental effects of the compound are outweighed by the beneficial effects.

As used herein, the terms “prevent”, “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “administering” and “introducing” are used interchangeably herein and refer to the placement of the modulator of CD150/SLAM/SLAMF1 expression into a subject by a method or route which results in at least partial localization of a gene product modulator, e.g., a CD150/SLAM/SLAMF1 modulator at a desired site. Such a modulator for administration to a subject may be selected from a Th2 blocking agent, e.g. an antibody, as further described herein. The modulator, cell population or pharmaceutical formulation of the present invention can be administered by any appropriate route which results in an effective treatment in the subject. In some embodiments, administering is not systemic administration. In some embodiments, administration includes contacting a specific population of T cells ex vivo with a target gene or gene product modulator, e.g., a CD150/SLAM/SLAMF1 modulator as disclosed herein, and administering the treated specific T cell population to a subject. For example, in some embodiments, a CD8 T-cell population is contacted with a target gene or gene product modulator, e.g., a CD150/SLAM/SLAMF1 activator, and the activator treated CD8+ T-cells are administered to a subject, e.g., a subject in need of treatment, such as, for example, a subject with a chronic immune diseases, e.g., a chronic infection and/or cancer.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration”, “administered systemically”, “peripheral administration” and “administered peripherally” as used herein mean the administration of a CD150/SLAM/SLAMF1 modulator such that it enters the animal's system and, thus, is subject to metabolism and other like processes, for example, subcutaneous administration

The immune cells of the present invention may be used for adoptive cell transfer. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma and colorectal carcinoma, as well as patients with CD19-expressing hematologic malignancies (Kalos et al., (2011) Science Translational Medicine 3 (95): 95ra73).

Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCRα and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3 or FcRy (scFv-CD3 or scFv-FcRy; see U.S. Pat. Nos. 7,741,465; 5,912,172; 5,906,936).

Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3-chain, CD97, GDI 1a-CD18, CD2, ICOS, CD27, CD154, CD5, OX40, 4-1BB, or CD28 signaling domains (for example scFv-CD28-4-1BB-CD3t or scFv-CD28-OX40-CD3; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3.zeta. and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with gamma-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ) CAR T cells of this kind may for example be used in animal models, for example to treat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction).

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD⁺ T-cells (see PCT Patent Publications: WO2013176915, WO2014059173, WO2014172606, WO2014184744, and WO2014191128). Editing may result in inactivation of a gene.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor a-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

A TIL product, for use in treatment, comprising TILs expressing SLAM/SLAMF1/CD150 may be obtained by enriching for cells expressing SLAM/SLAMF1/CD150 from cells originating from a subject or by engineering cells to express SLAM/SLAMF1/CD150.

T-cells including tumour infiltrating lymphocytes (TILs) expressing SLAM/SLAMF1/CD150 of the present invention may be ex vivo either from a patient's own peripheral blood (autologous), or in the setting of a haematopoietic stem cell transplant from donor peripheral blood (allogeneic), or peripheral blood from an unconnected donor (allogeneic). In these instances, T-cells expressing SLAM/SLAMF1/CD150 and, optionally, a CAR and/or TCR, are generated by introducing DNA or RNA coding for the SLAM/SLAMF1/CD150 and, optionally, a CAR and/or TCR, by one of many means including transduction with a viral vector, transfection with DNA or RNA.

For purposes of the inventive methods, wherein cells are administered to the patient, the cells can be T cells that are allogeneic or autologous to the patient.

A method for the treatment of disease relates to the therapeutic use of a vector or cell of the invention. In this respect, the vector, or T-cell may be administered to a subject having an existing disease or condition in order to lessen, reduce or improve at least one symptom associated with the disease and/or to slow down, reduce or block the progression of the disease. The method of the invention relates to increasing the tumour reactivity of T cells, such as T cells that are present in and make up the vast majority of a TIL cell population. Increased anti-tumour activity has been demonstrated by the data herein which shows that T-cells with increased SLAM/SLAMF1/CD150 correlates with improved clinical response in cancer patients treated with these cells. These cells may be considered tumour reactive T-cells. It is not known why increased SLAM expression correlates with improved clinical response (referred to herein as increased tumour reactivity), for example, it may be that SLAM is involved in increasing tumour killing or SLAM may be involved in promoting T-cell persistence.

Through this disclosure and the knowledge in the art, the DNA targeting agent as described herein or nucleic acid molecules encoding or providing components thereof may be delivered by a delivery system herein described both generally and in detail.

Vector delivery, e.g., plasmid, viral delivery: In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10¹⁰ particles, more preferably at least about 1×10⁸ particles (e.g., about 1×10⁸-1×10¹¹ particles or about 1×10¹⁰-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1 10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

The invention also relates to methods for enriching, expanding or treating a tumour reactive T cell population as described herein, for example a TIL population, that comprise the step of exposing the T cells to a modulator of CD150/SLAM/SLAMF1, e.g. one that increases CD150/SLAM/SLAMF1 gene expression or enhances CD150/SLAM/SLAMF1 protein activity. As explained herein, the methods for enriching, expanding or treating a tumour reactive T cell population as described herein may comprise the step of expanding the cells. In further aspects of the invention, the step of expanding the cells may include adding the modulator; e.g. one that increases CD150/SLAM/SLAMF1 expression. The inventors have found that cytokines can enhance CD150/SLAM/SLAMF1 gene expression. The modulator is thus preferably selected from one or more cytokine, for a combination of example 2, 3, 4, 5, 6, 7, 8 9 or 10 cytokines. Alternatively or additionally, the modulator may be a Type 2 T helper (Th2) blocking agent. The modulator; i.e. cytokine as described herein, may also have advantageous effects on other characteristics of the T cell population, such as expansion and the number of CD8+ T cells.

For example, the invention relates to a method for ex vivo or in vitro expansion of tumour-reactive T cells, such as TILs, for use in adoptive cell therapy, comprising culturing the T cells to produce expanded T cells in a culture medium wherein the culture medium comprises a modulator that increases CD150/SLAM/SLAMF1 expression.

In another example, the method is a method of obtaining a cell population enriched for tumour-reactive T-cells, the method comprising:

(a) obtaining a bulk population of T cells from a tumour sample, for example a TIL population;

(b) culturing the cells in the presence of a modulator that increases CD150/SLAM/SLAMF1 gene expression; and

(c) separating the cells selected in (b) from unselected cells to obtain a cell population enriched for tumour-reactive T cells.

The invention also relates to a method for preparing an enriched and expanded cell population of tumour reactive T-cells for use in cancer therapy comprising identifying and/or obtaining a cell population expressing CD150/SLAM/SLAMF1 and expanding the cell population wherein the cells are exposed to a modulator that increases CD150/SLAM/SLAMF1 expression. The cells may be exposed to a modulator that increases SLAM expression as part of the expansion step or in a separate step. The modulator may be a cytokine. For example, the modulator may be a single cytokine or a combination of two or more cytokines, for example 3, 4, 5, 6, 7, 8 9 or 10 cytokines.

In particular, the cell population comprises one of more of CD8+ and CD4+ cells. As explained elsewhere herein, the cells may be used in adoptive cell therapy. Expression of CD150/SLAM/SLAMF1 upon exposure to the CD150/SLAM/SLAMF1 modulator, i.e. the cytokine or combinations described herein, is increased compared to a reference point, for example compared to CD150/SLAM/SLAMF1 expression in untreated cells, i.e. cells that have not been exposed to the modulator. Expression is increased by at least 5%, 10%, 15%, 20% or 25%, for example by 5%, 6%, 7%, 8%, 9%, 10%, 11%, 12%, 13%, 14%, 15%, 16%, 17%, 18%, 19%, 20%, 21%, 22%, 23%, 24% or 25%.

As mentioned above, in these methods, the modulator of CD150/SLAM/SLAMF1 expression is advantageously selected from a cytokine; e.g. a single cytokine or a combination of 2 or more cytokines. The cytokine may be used in combination with another agent as explained below. Cytokines are a cell-signaling group of low molecular weight extracellular polypeptides/glycoproteins synthesized by different immune cells, mainly, by T cells, neutrophils and macrophages, which are responsible to promote and regulate immune response (i.e. activity, differentiation, proliferation and production of cells and other cytokines). These polypeptides act on signaling molecules and cells, stimulating them toward sites of inflammation, infections, traumas, acting on primary lymphocyte growth factors and other biological functions. Cytokines include interleukins (IL) and interferons (IFN).

In one embodiment, the cytokine or combination of cytokines comprises a cytokine selected from one that are induces in vitro differentiation to Type 1 T helper (Th1) (“Th1 skewing”). Th1 and Type 2 T helper (Th1)Th2 result from the differentiation of CD4+ T cells which respond to polarizing signals for Th cell differentiation. Th1 and Th2 are characterised by their mutually exclusive expression pattern of cytokines. Th1 cells produce IFN-γ and IL-2, whereas Th2 cells produce IL-4, IL-5, IL-9, IL-10 and IL-13. Functionally, Th1 responses are required for the clearance of intracellular infections, and Th2 responses are required for the clearance of helminth infection. The failure to generate the appropriate Th cell responses is often the cause of chronic infectious diseases. Under autoimmune conditions, polarized Th1 and Th2 responses are associated with organ-specific autoimmune diseases and allergies, respectively.

For example, IL-12 induces in vitro differentiation to Th1 (“Th1 skewing”) whereas IL-4 induces differentiation to in vitro differentiation to Th2 (“Th2 skewing”). Th1 skewing cytokines are thus for example selected from IL-12, IL-18, IL-7 or a combination thereof, preferably in combination with a Th2 blocking reagent, such as αIL4.

Exemplary modulators are that are Th1 skewing and which can be used according to the invention are the cytokines IL-2, IL-15, IL-18, IL-12, IL-23, IL-27, IL-35, IL-39, IL-18, IL-36, IL-37, IL-38, IFN-alpha, IFN-beta, IFN-gamma.

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rpi) chain binds to the p40 subunit of IL-12, providing the primary interaction between IL-12 and its receptor. However, it is IL-12p35 ligation of the second receptor chain, IL-12RP2, that confers intracellular signaling. IL-12 signaling concurrent with antigen presentation is thought to invoke T cell differentiation towards the T helper 1 (Th1) phenotype, characterized by interferon gamma (IFNγ) production. Th1 cells are believed to promote immunity to some intracellular pathogens, generate complement-fixing antibody isotypes, and contribute to tumor immunosurveillance. Thus, IL-12 is thought to be a significant component to host defense immune mechanisms. IL-12 is part of the IL-12 family of cytokines which also includes IL-23, IL-27, IL-35, IL-39.

Interleukin 6 (IL-6) belongs to a distinct family of cytokines that uses a cytokine specific receptor chain paired with a common gp130 receptor for signaling. IL-6, which induces phosphorylation of STAT3, similarly to IL-21, can stimulate proliferation of CD8+ T cells in synergy with IL-7 or IL-15. IL-6 has been reported to promote CD8+ lymphocyte effector functions and protect T cells from apoptotic death.

Interleukin-18 (IL-18) is a proinflammatory cytokine that belongs to the IL-1 cytokine family, due to its structure, receptor family and signal transduction pathways. Related cytokines include IL-36, IL-37, IL-38.

Interleukin 21 (IL-21) is a member of the common γ-chain (γc) receptor cytokine family, and has been reported to regulate the development and function of various T cell subsets. Common-γ chain receptors consist of a cytokine-specific subunit and a shared γ-chain CD132 subunit. The gamma-chain subunit associates with different cytokine-specific receptor subunits to form unique heterodimeric receptors for IL-4, IL-7, IL-9, and IL-21, or associates with both IL-2/IL-15R beta and IL-2R alpha or IL-15R alpha to form heterotrimeric receptors for IL-2 or IL-15, respectively. IL-21 signals through STAT3 to promote functional maturation of memory CD8+ T cells. IL-21 can synergize with IL-7 to induce the expansion of antigen-activated CD8+ cells and augment anti-tumor activity by production of Th and inflammatory cytokines.

The cytokines, i.e. interleukins and interferons, referred to herein, include human and mammalian forms, conservative amino acid substitutions, glycoforms, biosimilars, and variants thereof. The term also encompasses pegylated forms.

Thus, in one embodiment, the cytokine is selected from IL-2, IL-15, IL-18, IL-12, IL-23, IL-27, IL-35, IL-39, IL-18, IL-36, IL-37, IL-38, IFN-alpha, IFN-beta, IFN-gamma or a combination thereof. In one embodiment, if a single cytokine is used, the cytokine is not one that is commonly used in the expansion of T cells, e.g. IL-2.

In one embodiment, the modulator comprises a combination of cytokines, in particular a combination of Th1 skewing cytokines or a combination where at least one cytokine is a Th1 skewing cytokine. In one embodiment, the modulator comprises a combination of an IL-12 family cytokine, e.g. IL-12, with another cytokine; a combination of IL-2 with another cytokine; a combination of IL-7 with another cytokine; a combination of IL-18 with another cytokine or a combination of IL-15 with another cytokine. In one embodiment, the combination is selected from IL-7+IL-15, IL-2+IL-7+IL-15, IL-2+IL-12, IL-2+IL-4, IL-2+IL-18, IL-2+IL-12+IL-7+IL-15, IL-2+IL-12+IL-7+IL-15+IL-6, IL-2+IL-12+IL-7+IL-15+IL-21, IL-2+IL-12+IL-7+IL-15+IL-6+IL-21, IL-7+IL-15+IL-6, IL-7+IL-15+IL-21, IL-7+IL-15+IL-6+IL-21, IL-2+IL-12+IL-6, IL-2+IL-12+IL-21, or IL-2+IL-12+IL-6+IL-21.

The modulator may further include a Th2 blocking reagent, such as an antibody. The antibody may be selected from anti-IL-4 (αIL4), anti IL-4R (αIL4R), anti IL-5R (αIL5R), anti IL-5 (αIL5), anti-IL13R (αIL13R) or anti-IL13 (αIL13). In one embodiment, the antibody is αIL4. In one embodiment, the antibody is selected from Mepolizumab, Resilizumab, Benralizumab, Tralokinumab, Lebrikizumab or Dupilumab.

In one embodiment, the modulator comprises a cytokine selected from IL-2, IL-15, IL-18, IL-12, IL-23, IL-27, IL-35, IL-39, IL-18, IL-36, IL-37, IL-38, IFN-alpha, IFN-beta, IFN-gamma or a combination of two or more cytokines thereof in combination with a Th2 blocking reagent, e.g. an antibody selected from anti-IL-4 (αIL4), anti IL-4R (αIL4R), anti IL-5R (αIL5R), anti IL-5 (αIL5), anti-IL13R (αIL13R) or anti-IL13 (αIL13) In one embodiment, the antibody is anti-IL-4 (αIL4).

In one embodiment, the modulator comprises a combination selected from one of IL-2+αIL4; IL-12+αIL4; IL-2+IL-12+αIL4; IL-2+IL-12+IL-7+IL-15+αIL4; IL-7+αIL4; IL-15+αIL4. In one embodiment, the modulator is selected from a modulator that comprises IL-2+IL-7+IL-15, IL-2+IL-12, IL-2+IL-18, IL-2+IL-12+IL-7+IL-15+IL-21, IL-7+IL-15+IL-21, or IL-2+IL-12+IL-21. In particular, as shown herein, CD4+ cells treated with IL-2+IL-7+IL-15 and IL-2+IL-12 have enhanced SLAM expression and CD8+ cells treated with IL-2+IL-18 have enhanced SLAM expression.

In some embodiments, the cells may be exposed to the modulator for about 1 hour to 1, 2, 3, or 4 weeks, e.g. abut 1 hour, about 6 hours, about 12 hours, about 24 hours, about 36 hours, about 48 hours, about 3 days, about 5 days, about 1 week, about 10 days, about 2 weeks, about 3 weeks, or about 4 weeks.

In another embodiment, IL-12 is added at the beginning of the expansion step, either alone or in combination with another cytokine, such as IL-2, but is then replaced another cytokine or a combination of cytokines for the remainder of the expansion, such as with a combination of IL-2+IL-7+IL-15, IL-7+IL-15 or a combination of a cytokine, such as IL-15, IL-7 and a Th2 blocking antibody, such as αIL4; e.g. IL-15+αIL4. Without wishing to be bound by theory, it is assumed that brief exposure to IL-12 stimulates SLAM expression, but avoids the adverse toxicity effect of IL-12 exposure.

For example, exposure to IL-12 may be provided to the cell population for a duration of 1-96 hours; e.g. 1 to 12, e.g. 1, 2, 3, 4, 5, 6, 7, 8, 9, 10, 11; 1 to 24, 1 to 36, 1 to 48, 1 to 60, 1 to 72, 1 to 84 hours, before it is replaced with another cytokine, for example IL-2+IL-7+IL-15.

The cytokines may be provided in the following concentrations 5-150 ng/ml, for example 5, 10, 20, 25, 30, 40, 50, 60, 70, 80, 90, 100, 110, 120, 130, 140, 150 ng. In some embodiments, the concentration may be increased or decreased over time; e.g. over 1-96 hours; e.g. 1 to 12, 1 to 24, 1 to 36, 1 to 48, 1 to 60, 1 to 72, 1 to 84 hours.

When a combination of cytokines/a combination of cytokines with an antibody is used as explained above, the components of the combination are provided simultaneously or separately, but preferably provided simultaneously.

The cells may be exposed to the modulator prior to the cell expansion step or during cell expansion.

In one embodiment, the modulator is produced by an artificial antigen presenting cell (aAPC) and the tumor reactive T cells are co-cultured with the antigen presenting cell. Thus, the invention also relates to a method for method for preparing an enriched and expanded cell population of tumour reactive T-cells for use in cancer therapy comprising identifying and/or obtaining a cell population expressing CD150/SLAM/SLAMF1 and expanding the cell population wherein the T cells are co-cultured with a population of artificial antigen presenting cell that produce cytokines. For example, the cytokines are expressed as a secreted or membrane anchored form on a cell line which is then used for expansion of cells.

In an embodiment, the invention provides a method of expanding a population of tumour reactive T cells, e.g. tumor infiltrating lymphocytes (TILs), the method comprising the (b) contacting the population of tumour reactive T cells with the population of aAPCs that express a cytokine, e.g a combination of cytokines, in a cell culture medium. The cytokine can be selected from Th1 skewing cytokines as explained above. In a further step, the method may comprise exposing the cells to a Th1 blocking agent as described above, e.g. an antibody. The method may be an in vitro or an ex vivo method.

As explained elsewhere, the T cell may be a population of a population of tumour infiltrating lymphocytes (TILs) from a tumour biopsy, lymphnode or ascites; and/or a population of T-cells engineered to express a CAR and/or a TCR a population of T-cells isolated from blood. The T cells are tumour reactive and useful for adoptive T cell therapy. The T cell may a CD4+ or a CD8+ T cell.

aAPCs developed for use in the expansion of TILs and focus so far has been on the well-established K562 cell line The APC may thus comprise a K562 cell, for example transduced using a suitable vector, e.g. a lentiviral vector (LV). The aAPC may be modified to express one or more costimulatory molecules.

In another embodiment of the methods, the T cells are engineered to express a cytokine receptor which provides cytokine signaling upon engagement of another drug or cytokine. Cytokine signaling refers to cytokine Th1 skewing signaling as explained above. The receptor provides a Th1 signal in response to a Th2 cytokine. The receptor may, for example, be a IL-4-IL-2 receptor or IL-4-IL-12 receptor.

The invention further relates to the use of cytokine, cytokine combination or combination of a cytokine and Th2 blocking agent as set out above in increasing expression of SLAM, in particular in an isolated T cell population.

The invention further relates to a method for increasing expression of CD150/SLAM/SLAMF1 in tumour reactive T-cells for use in cancer therapy comprising identifying and/or obtaining a cell population expressing CD150/SLAM/SLAMF1 and expanding the cell population in the presence of a cytokine that increases expression of CD150/SLAM/SLAMF1.

The invention also relates to a method for identifying an agent for increasing SLAM expression in isolated ex vivo expansion of T cells, e.g. tumour-infiltrating lymphocytes, for use in adoptive cell therapy, comprising contacting tumour-infiltrating lymphocytes with a candidate modulator for the ability to upregulate the expression of SLAM in T cells, for example CD4+ and CD8+ T cells; screening the effect of the modulator on the expression of SLAM in the cells, identifying a modulator that increases the expression of SLAM in the cells.

Identification of a candidate modulator that increases expression of SLAM in T cells identifies an agent that can be used in for ex vivo expansion of tumour reactive T cells, such as T cells in TIL populations, for use in ACT.

Further details of aspects and embodiments of the invention are below.

There now exists irrefutable evidence that the immune system can mount an effective response to many cancer types. This has led to the development of a number of cancer immunotherapies. These can be broadly classified into cell-based immunotherapies, and non-cell based. Non-cell-based immunotherapies have shown some exciting results. In particular the 30 use of checkpoint blockade antibodies such as anti-PD1 (Nivolumab; Pembrolizumab), and PDL1 (Atezolizumab), or anti-CTLA4 (Ipilumumab) have had remarkable results in the clinical setting for advanced metastatic melanoma. Cell-based immunotherapies for cancer have had equally exciting results in early trials with CD19-CAR T-cells targeting B-cell malignancies in particular showing encouraging results.

TIL therapy is generally a simpler approach in that it requires no genetic modification and thus is appealing. In melanoma response rates of around 50% are generally observed. A trial in cervical cancer also showed 33% response rate. There are also a number of ongoing trials for other cancer indications including ovarian cancer.

Cancer response is measured using RECIST guidelines (version 1 published in 2000) and more recently updated in RECIST guidelines 1.1 (Eisenhauer et al 2009). Enabling the uniform assessment of the change in tumour burden, which is an important feature of the clinical evaluation of cancer therapeutics: both tumour shrinkage or no detectable disease i.e. the objective response (CR+PR), no change (stable disease (SD)) and disease progression (PD) which are useful endpoints in clinical trials to determine therapeutic efficacy and patient prognosis. The critical change in RECIST 1.1 was that it allows tumours to increase in size due to inflammation prior to shrinkage which is critical to allow therapeutics such as TIL to function where otherwise these therapies may be termed a failure.

The present invention provides a method for the prognosis of the outcome of treatment with Tumour Infiltrating Lymphocyte therapy in a patient, which novel method is based on the detection and/or the quantification of one or more biological markers, e.g. on the CD4+ and/or CD8+ Tumour Infiltrating Lymphocytes in the tumour, in the product during the TIL manufacturing process or in the TIL product prior to infusion.

By flow cytometric analysis of the TIL product the applicants have found a cell molecular marker expressed on the CD4+ and/or CD8+ T-cells within the TIL product which correlates with the response seen in the patient upon infusion.

The marker described herein which has been shown by the present inventors to correlate with patient response is SLAM (CD150).

It has been found according to the invention that there is a correlation between expression of the cell surface marker and the outcome of treatment with TIL.

The receptor identified is SLAM (Signaling lymphocyte activation molecule/SLAMF1/CD150). SLAM is a name given to a particular member of the SLAM family of receptors, referring 30 specifically to the SLAM family receptor 1 (SLAMF1). The SLAM family contains a number of other members including SLAMF3 (CD229), SLAMF4 (CD244) and SLAMF7 (CRACC/CD319). Most of these receptors are self-ligands, that is they bind to one another across haematopoietic cells. The cytoplasmic domain of SLAM family receptors contains immunoreceptor tyrosine-based switch motifs which associate with SAP (in T-cells) or EAT-2 (in NK-cells) protein adaptors. The role of SLAM family receptors remains somewhat unresolved. They can assist in immune responses in an activatory role or an inhibitory role, depending on the SLAM family receptor in question and in which cell it is expressed. CD150 itself has proven costimulatory function. SLAM engagement induces a TH1 phenotype of cytokines dominated by IFNγ and it has thus been suggested that manipulation of SLAM may be beneficial for TH2 polarized disease (Quiroga et al 2004). Furthermore, it has been noted that SLAM is expressed to a higher degree in TH1 cells as compared to TH2 cells (Hamalainen et al 2000) which may partly explain the observation as to why it induces TH1 like cytokines. The final interesting point to make with regards CD150 is that it is the primary viral receptor for the measles virus (Erlenhoefer et al 2001). This has been exploited for cell therapy purposes by using lentiviruses which are pseudotyped with the measles virus envelope to more specifically target lentiviruses to T-cells (Frecha et al. 2011)

Thus, a first object of the present invention consists of an in vitro method for the prognosis of patients who may receive Tumour Infiltrating Lymphocyte (TIL) therapy for cancer; the method comprises the following:—

i) quantifying in a sample of tumour digest, TIL material during the manufacturing process or TIL product, from said patient, at least one biological marker on the TIL; and

ii) comparing the value obtained at step i) for said at least one biological marker with a predetermined reference value for the same biological marker; which predetermined reference value is correlated with a specific prognosis of progression of said cancer.

Although there are previous examples of molecular markers on TIL which appear to correlate with clinical efficacy (for example BTLA), none have described SLAM. Radvanyi et al. 2015 have described the proportion of CD8+ T-cells within TILs as being associated with good clinical outcome. This is confounded by the observation that BTLA can act as a negative regulator of T-cell activity (Watanabe et al. 2003)

TIL products enriched for CD8+ cells does not confer a survival advantage over mixed CD4+ and CD8+ cells (Dudley et al., 2010). This is largely assumed to be because the presence of CD4+ helper T-cells assists the CD8+ T-cell survival and engraftment. Furthermore, there is evidence that CD4+ cells can assist in direct recognition and killing of tumour cells (Tran et al., 2014).

Radvanyi et al. (2015) also described the expression of BTLA as having favorable association with clinical benefit. The expression of BTLA has been associated with less differentiated T-cells with enhanced survival properties (Haymaker et al., 2015). However, paradoxically it has also been shown that the presence of more differentiated effector memory T-cells within TIL infusion products is associated with favorable outcome. Effector memory T-cells are usually marked by the co-expression of CD62L or CCR7 combined with CD45RO or CD45RA such that effector memory cells can have the following phenotypes: CD62L−/CD45RO+, CD62L−/CD45RA−, CCR7−/CD45RO+ or CCR7−/CD45RA−.

There are also a number of publications and prior art works describing associations with various cell surface markers on TIL in vivo with clinical outcome, but this is not associated with TIL manufacture, rather the analysis of tumour biopsies. Examples include patent US20090215053A1—Vitro Method for the Prognosis of Progression of a Cancer and of the Outcome in a Patient and Means for Performing Said Method

As intended herein, Tumour Infiltrating Lymphocytes are a) CD45+ Cells isolated directly from the tumour sample of a patient with cancer via physical or enzymatic disaggregation, b) CD45+ cells isolated from lymph nodes from said patient c) CD45+ cells isolated from ascites (otherwise termed tumour associated lymphocytes). The TIL remain as such throughout the TIL manufacturing process with a propensity for them to obtain a slightly different phenotype wherein they remain CD45+ but the proportion of CD3+ cells increases as the cells are cultured in IL-2 and activated with irradiated feeder cells or an alternative TIL expansion system. These CD45+/CD3+ cells are termed T-cells or T-lymphocytes. As such the term ‘TIL’ encompasses any CD45+ cell from the point of surgery to the point of infusion back to the same patient.

The analysis of said markers (SLAM/SLAMF1/CD150) may be performed by one of several mechanisms. In the first instance flow cytometry may be performed. In this instance the cells may be stained with antibodies to SLAM to determine its presence or absence. Furthermore other antibodies may be incorporated into the staining panel to look at defined populations of cells within the TIL. For example the cells may be counterstained with antibodies to CD62L, CD45RO, CD4 and/or CD8.

The said markers (SLAM/SLAMF1/CD150) may be otherwise exploited in such a fashion as to enrich cells expressing said markers in an effort to improve the efficacy of the final product. For example flow cytometry can be taken advantage of to isolate cells expressing said markers by using specific antibodies (anti-SLAM) or recombinant (r) protein (for example r-SLAM) conjugated to fluorophores or other direct or indirect selectable markers.

Alternatively, other technologies for isolating cells may be performed. For example Miltenyi MACS magnetic technology, Invitrogen Dynal technology or Stem Cell Technologies EasySep technology may be used to isolate cells expressing said markers.

Alternatively yet, antibodies (or antibody fragments thereof) or recombinant protein either immobilized to plates, beads or other solid matrix, or expressed on an artificial antigen presenting cell platform may be used to enrich cells expressing said markers. In such examples the antibodies or recombinant protein may be found alone or in combination with an antibody or other activation platform which induces a primary activation signal to T-cells (examples include but are not limited to: Phytohaemagglutinin, anti-CD3 antibody, Peptide-major histocompatability antigen complex, phorbol myristate acetate).

The goal of all of the above is then to stratify patients based on expression of said markers, and where possible to isolate and/or enrich the therapeutic cells via one or more methods described above.

As intended herein, multiple methods are available in the art to assess the presence of the biomarker either using: (a) direct methods where biomarker binding moieties are bound to secondary reporting moieties in a single or multiple step process such as biomarker binding antibodies conjugated to a reporter system that can be detected such as those commonly used in flow cytometry. Microscopy or proteomic gel chromatography; or (b) indirect methods where biomarker encoding nucleic acid are quantified such as quantitative PCR. Where the method of choice is analysing cells on a single cell and population based method wherein cells are loaded with antibodies to defined surface markers which may be directly or indirectly coupled to fluorophores, which emit light at defined wavelengths which can be detected by components of the flow cytometer machine. Herein the term flow cytometry is preferred to the more commonly used but incorrect term FACS which is a trademark of Becton Dickinson flow cytometry machines. As such any flow cytometer may be used for the analysis (Becton Dickinson [BD], Miltenyi, Acea etc) for the purposes of this method but other methods may be employed.

Preferably, when step a) consists of the expression analysis of one or more genes, i.e. one or more pertinent biological markers, then the quantification of the expression of the said one or more genes is performed from the whole tumor tissue sample.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and tables described below.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1—Analysis of Expression of SLAM/CD150 in Tumour Infiltrating Lymphocytes

Metastatic melanoma tumour biopsies were taken from patients and brought to the lab where they were first cut into fine pieces using a scalpel, and then digested to a single cell suspension using a mixture of collagenase and DNase.

The cell suspension was seeded at approximately 1×10⁶ viable CD3+ cells per well of a 24-well plate in complete media with the addition of 3000 IU/ml IL-2. Cultures were monitored for cell growth and split accordingly as needed to maintain a healthy culture.

The TIL are grown for up to 21 days or until the CD3+ cell count was greater than 30×10⁶ after which the cells were washed and frozen. Prior to freezing a sample was taken for flow cytometric analysis. At this point the sample is referred to as Pre-REP. Before the cells are ready for infusion they need to be expanded, preferably to numbers of 10⁹ or more, more preferably in excess of 1×10¹⁰. To achieve this the TIL undergo a rapid expansion protocol (Dudley et al., 2003). In brief, the TIL cells were defrosted 1 to 3 days prior to expansion. The TIL are then mixed at a 1:50 to 1:1000 ratio with irradiated auto/allo-geneic PBMC feeder cells with the addition of 10-50 ng/ml OKT3. The TIL were then expanded for a period of 2 weeks after which they are referred to as Post-REP TTL. Prior to infusion of the Therapeutic TIL product the (i.e. Post-REP TIL) the patient underwent pre-conditioning chemotherapy consisting of Fludarabine and Cyclophasphamide. The Post-19 REP TIL are issued to the patient and given as a single infusion prior to a number of doses of IL-2.

At the point of Pre-REP and Post-REP the TIL cells were analysed by flow cytometry. In brief, three samples of 1-2×10⁵ cells were washed in PBS and then incubated with 50 μl of 1:400 dilution of Fixable Viability Dye eFluor 450 for five minutes at room temperature in the dark. Cells were washed twice with 150 μl of PBS and then resuspended in 50 μl cold PBS supplemented with 2 mM EDTA and 0.5% foetal calf serum (PEF). To each sample antibodies were added as indicated in the table below for 30 minutes at 4° C.

TABLE 1

| | | | Sample | | |
|---|---|---|---|---|---|
| Antibody/Dye | Supplier | Concentration | 1 | 2 | 3 |
| Fixable Viability Dye | ebioscience | 1:400 | y | y | Y |
| eFluor 450 | | | | | |
| CD45RO FITC | Miltenyi | 1:25 | y | y | Y |
| CD8 PE Vio770 | Miltenyi | 1:25 | y | y | Y |
| CD4 APC Cy7 | Biolegend | 1:25 | y | y | Y |
| CD62L APC | Biolegend | 1:25 | y | y | Y |
| mIgG1 PE | Biolegend | 1:25 | y | — | — |
| mIgG1 PerCP eFluor 710 | ebioscience | 1:25 | y | — | — |
| SLAM PE | Miltenyi | I 1:25 | — | y | — |
| GITR PerCP eFluor 710 | ebioscience | 1:25 | — | y | — |

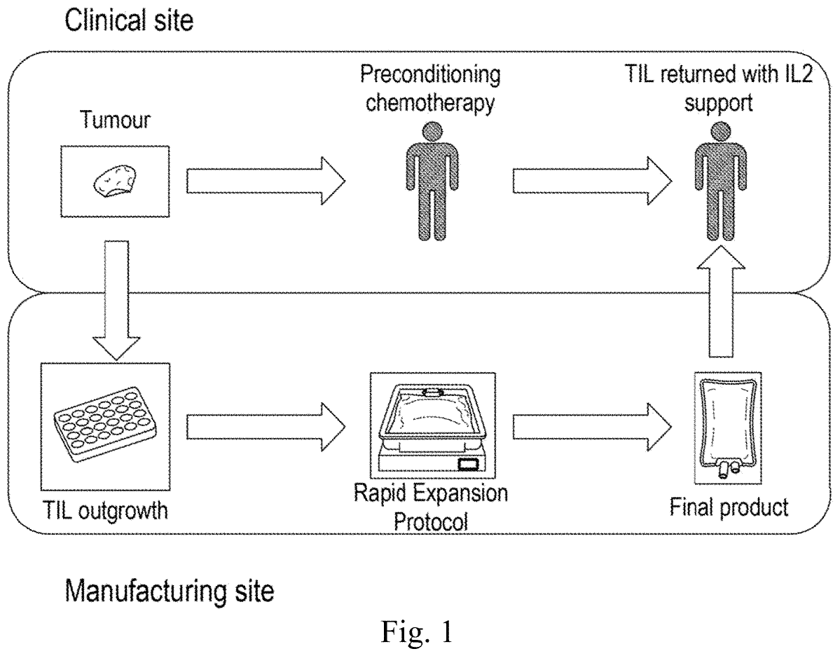

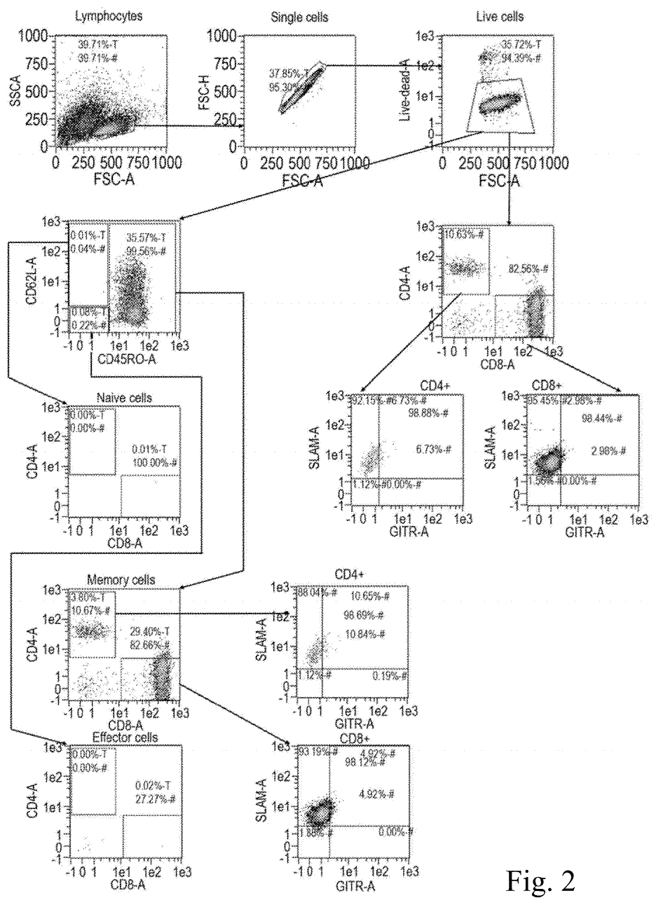

After incubation the cells were washed twice with 150 μl cold PEF and finally resuspended in 200 μl PBS. Cells were acquired on a MACSQuant analyser and data analysed using MACSQuantify software (FIG. 1). When comparing TIL at Pre-REP and Post-REP Applicants found enhanced expression of SLAM in the CD4+ and CD8+ TIL at the Pre-REP stage compared to the Post-REP stage. In particular Applicants observed two distinct populations of SLAM expression in the CD4+ Post-REP TIL suggesting a group of patients who were SLAM-high and SLAM-low. In particular the SLAM expression tended to be largely restricted to the memory cell populations in the CD4+ and CD8+ populations, with lower expression in the naïve and effector populations. The naïve and effector populations form a small portion of the overall TIL products.

When Applicants analyzed the proportion of SLAM+ cells with respect to patient response in cutaneous melanoma, a significantly increased proportion of CD4+ T-cells which were SLAM+ in the patients who had stable disease (p=0.0252) or responders (p=0.0113), compared to those who had progressive disease (‘progressors’) were found. In the CD8+ population the significant difference observed was between those patients who had a response to treatment and those who had progressive disease (p=0.0248). The significant differences were mainly restricted to the differences seen in the memory cell populations. In CD4+ T-cells the memory cell population had a significant difference between the patients with stable disease and progressors (p=0.0384) and between progressive disease and responders (p=0.0221). In the CD8+ memory T-cells Applicants saw a significant difference between patients with progressive disease and stable disease (p=0.0348) and between those with progressive disease and responders (p=0.0169).

Following treatment with the TIL infusion product the patients were evaluated to measure the tumour size according to response evaluation criteria in solid tumours (RECISTv1.1) measurements. The best response was determined for each patient according to the latest RECIST 1.1 guidelines (Schwartz et al. 2016). Applicants plotted overall survival of patients against duration of response (FIG. 4). Applicants used two cut-offs for SLAM-high and SLAM-low: 25% and 40% of all CD4+ TIL in the final product respectively. Applicants found that patients who had >25% SLAM+ cells had enhanced survival compared to those that had <25% SLAM+ cells (Median 21.3 months v 2.4 months respectively, with a significant difference of p=0.009 by Wilcoxon test, and a plateau of 44% (FIG. 4A). Equally Applicants found that patients who had >40% SLAM+ cells had enhanced survival compared to those that had <40% SLAM+ cells (Median Not Reached v 4 months respectively, with a significant difference of p=0.027 by Wilcoxon test, and a plateau of 55% (FIG. 4A).

### Example 2—Modulation of SLAM Expression in T-Cells and TIL

Applicants first assessed the impact of SLAM expression in TIL on the viability and functional response of TIL towards matched tumour (TIL032 and TIL054). To this end Applicants took two TIL infusion products and sorted a SLAM-high and -low population using anti-SLAM antibodies and a flow sorter. Following an overnight rest period the cells were co-cultured with their autologous tumour line, or left in wells unstimulated, for 16 h and then the viability determined using DRAQ7 (FIG. 5A) and the proportion of cells producing IL-2, IFNγ and TNFα was determined using flow cytometry (FIG. 5B). Applicants found that TIL032 and TIL054 SLAM-high cells had 30 increased viability compared to SLAM-low or unsorted cells (FIG. 5A). When mixed with their tumour the effect was less obvious, but apparent in TIL054. When Applicants assessed cytokine production, elevated cytokine responses were found when the TIL were cultured with their matched tumour. This was most obvious when looking at production of TNFα. However, Applicants found that the TNFα response from the SLAM-high sorted populations were, on the whole, greater than from the SLAM-low sorted cells, with the exception of the CD8+ response to tumour in TIL054.

Next Applicants assessed impact of SLAMF1 siRNA on expression of SLAM in cell lines and T-cells as an approach to modulate SLAM expression. Applicants took the SLAM+ cell line Raji and two TIL samples (MRIBB011 a colorectal tumour TIL sample and the TIL032 infusion product from TIL032). The cells were plated in 96 wells (1×10⁵ per well) with 10 μM of self-delivery siRNA (Accell Human SLAMF1 siRNA SMARTpool, 5 nmol—[Dharmacon, Colo., USA]) in minimal media (RPMI+1% FCS+ITS, and for the TILs supplemented with 1000 IU/ml of IL-2). Controls were grown at the same conditions but without the siRNA. 76 h later 2×10⁵ cells (2× wells) from each condition were pooled and the Cells-to-CT™ 1-Step TaqMan® Kit (Invitrogen) was used.

Briefly the cells were spun down, washed with PBS (R/T) and pelleted again. Supernatant was removed and 49 μl Lysis solution+1 μl DNAse was added. They were incubated for 5 min at R/T and the 5 μl of STOP solution was added. Samples were left for 2 min at RT and then placed on ice until used. All above reagents are provided with the kit. The lysates are stable for up to 5 months at −80.

TaqMan reactions for SLAMF1 (Assay ID: Hs00234149_m1, Invitrogen) and GAPDH (Assay ID: Hs03929097_g1, Invitrogen) were set in triplicate, and the reaction volume was scaled down to 10 μl from the 20 μl suggested by the kit. 1 μl of the lysis was added in each reaction. A positive control reaction with already extracted RNA from untreated Raji cells was also set up to account for possible PCR inhibiting agents present in the cellular lysates.

Applicants observed an almost complete knock down of SLAM transcript in Raji cells (>99%) (FIG. 6A). In MRIBBO11 Applicants saw a 31% reduction of SLAM transcript; and in TIL032 Applicants saw a 57% increase in SLAM transcript. Protein expression was altered to a different degree (FIG. 6B). In Raji cells surface expression of the protein over the same time course decreased by 5.3%, in MRIBBO11 TIL by 30.8% and in TIL032 by 9.6%. Thus SLAM siRNA is a suitable means of modulating SLAM expression, although optimisation is required for each cell line tested to observe the optimal knock down in expression.

Next Applicants determined whether enhancement of SLAM expression in T-cells could provide an alternative approach to quantifying the impact of SLAM expression, rather than by knock-down 30 as achieved via siRNA. To this end a lentiviral expression construct was created in which SLAMF1 and a CD19 marker gene are driven by and EF1α promoter (FIG. 7A). Lentiviral particles were generated in HEK293T cells by transient transfection and then the resulting particles titrated on SLAM-negative Jurkat JRT3-T3.5 cells (FIG. 7B). Co-expression of SLAM and CD19 was demonstrated in the Jurkat cells.

### Example 3—Stimulation with Cytokines

Materials and Methods

Tumour Infiltrating Lymphocytes (TIL) Rapid Expansion.

TIL from 4 different donors with advanced cutaneous (donors 12, 32 and 43) and uveal (donor 42) melanomas were isolated by enzymatic tumour dissociation and grown for 14 days in RPMI media supplemented with 3000 units/ml of IL-2. The TILs were subsequently frozen and defrosted 2 days before the beginning of the rapid expansion stage. After defrosting they were placed for 2 days in RPMI supplemented with 200 units/ml of IL-2 and then were counted by Draq7 live dead staining at the MacsQuant (Milteny Biotech).

PBMCs were freshly isolated using ficol gradient from 4 healthy donors. The PBMCs were counted and irradiated and then mixed to make a pool of irradiated feeder cells for the TIL expansion.

The TILs were mixed with the irradiated PBMC feeders at a ratio of 1:200 (TIL:Feeders) and were placed in 24 Grex plates (100.000 TILs per 24 well) together with 7 ml of media. For each donor, 7 different expansion conditions were set up (table 2). The media was supplemented with the cytokines/antibodies detailed in Table 2 and was changed at days 6 and 12 (as per manufacturer instructions for the Grex plates). On Days 3 and 9 the media was just supplemented with the cytokines detailed in table 2 without a media change.

TABLE 2

| | Day 0 | Day 3 | Day 6 | Day 9 | Day 12 |
|---|---|---|---|---|---|
| 1 | IL-2 (3000 | IL-2 3000 | IL-2 3000 | IL-2 3000 | IL-2 3000 |
| | units/ml) + PHA | units/ml | units/ml | units/ml | units/ml |
| 2 | IL-2 (3000 | IL-2 3000 | IL-2 3000 | IL-2 3000 | IL-2 3000 |
| | units/ml) + | units/ml + | units/ml + | units/ml + | units/ml + |
| | IL-4 (100 | IL-4 100 | IL-4 100 | IL-4 100 | IL-4 100 |
| | ng/ml) + PHA | ng/ml | ng/ml | ng/ml | ng/ml |
| 3 | IL-2 (3000 | IL-2 3000 | IL-2 3000 | IL-2 3000 | IL-2 3000 |
| | units/ml) + | units/ml + | units/ml + | units/ml + | units/ml + |
| | IL-12 (25 | IL-12 | IL-12 | IL-12 | IL-12 |
| | ng/ml) + PHA | 25 ng/ml | 25 ng/ml | 25 ng/ml | 25 ng/ml |
| 4 | IL-2 (3000 | IL-2 3000 | IL-2 3000 | IL-2 3000 | IL-2 3000 |
| | units/ml) + | units/ml + | units/ml + | units/ml + | units/ml + |
| | αIL-4 | αIL-4 | αIL-4 | αIL-4 | αIL-4 |
| | (1 μg/ml) + PHA | 1 μg/ml | 1 μg/ml | 1 μg/ml | 1 μg/ml |
| 5 | IL-2 (3000 | IL-2 3000 | IL-2 3000 | IL-2 3000 | IL-2 3000 |
| | units/ml) + | units/ml + | units/ml + | units/ml + | units/ml + |
| | IL-12 (25 | IL-12 (25 | IL-12 (25 | IL-12 (25 | IL-12 (25 |
| | ng/ml) + | ng/ml) + | ng/ml) + | ng/ml) + | ng/ml) + |
| | αIL-4 | αIL-4 | αIL-4 | αIL-4 | αIL-4 |
| | 1 μg/ml + PHA | 1 μg/ml | 1 μg/ml | 1 μg/ml | 1 μg/ml |
| 6 | IL-2 3000 | +IL-7 10 | +IL-7 10 | +IL-7 10 | +IL-7 10 |
| | units/ml + | ng/ml + | ng/ml + | ng/ml + | ng/ml + |
| | IL-12 25 | IL-15 | IL-15 | IL-15 | IL-15 |
| | ng/ml + PHA | 10 ng/ml | 10 ng/ml | 10 ng/ml | 10 ng/ml |
| 7 | IL-2 3000 | +IL-7 10 | +IL-7 10 | +IL-7 10 | +IL-7 10 |
| | units/ml + | ng/ml + | ng/ml + | ng/ml + | ng/ml + |
| | IL-12 25 | IL-15 10 | IL-15 10 | IL-15 10 | IL-15 10 |
| | ng/ml + PHA | ng/ml + | ng/ml + | ng/ml + | ng/ml + |
| | | αIL-4 | αIL-4 | αIL-4 | αIL-4 |
| | | 1 μg/ml | 1 μg/ml | 1 μg/ml | 1 μg/ml |

At day 14 of rapid expansion total TIL was counted and 100.000 cells per condition were plated in 96 well round bottom plates and washed with PBS×2. They were then incubated with fixable viability dye eF450 (1 μL/mL) for 5 minutes at R/T, washed with PEF and stained with the following antibody mix or IgG isotype mix for 20 min at 4° C.

Antibody Mix:

### CD62L-APC

### CD4-APC-Cy7

### CD8-PE Vio777

### SLAMf1-PE

CD137-ef710

### CD45RO-FITC

Antibody Isotype Mix:

### CD62L-APC

### CD4-APC-Cy7

### CD8-PE Vio777

### IgG1-PE

CD137-ef710

### CD45RO-FITC

The cells were washed with PEF and then fixed with 4% PFA for 15 mins at 4° C. Following a wash they were resuspended with 100 μl of PEF per well and analysed using the MacsQuant (Milteny Biotech)

The remaining cells were placed in 3000 units/ml for 48 hours. Conditions 1, 2 and 3 were starved of IL-2 overnight at the end of 48 hours and then they were co cultured with K562s wild type and K562s expressing the OKT3 anti CD3 antibody. The co-culture was set up at a ratio of 1:2 (TIL: K562/K562 OKT3) for 5 hours in 96 well round bottom plates (100.000 TIL per well). During the co-culture Brefeldin and Monensin was added to the media together with CD107a-Viobright FITC.

Subsequently the cells were washed with PBS and incubated with fixable viability dye eF450 (1 μL/mL) for 5 minutes at R/T. The cells were washed with PEF and then fixed with 4% PFA for 15 mins at 4° C. Following a wash they were resuspended with 100 μl of PEF and split in 2 plates for antibody and IgG control staining.

The cells were then washed with perm/wash buffer and stained for the following mix of antibodies or IgG isotypes for 45 min at 4° C.

CYTOKINE Mix in Perm Wash:

### IL-2 PE-Cy7

### IFN-7 PE

### TNF APC-Cy7

CD2 eF710

IgG ISOTYPE Mix in Perm Wash:

### ISO PE-Cy7

### ISO APC-Cy7

### ISO PE

CD2 eF710

Following staining the cells were washed with perm/wash buffer (×2) and then resuspended in PEF containing (2 in 50) CD8 APC antibody. They were incubated for 20 min at 4° C. and then washed ×2 with PEF. They were resuspended in 100 μl per well and analysed using the MacsQuant (Milteny Biotech).

### Example 4—Analysis of the Effects of Cytokine Conditions on SLAM Expression and TIL Phenotype

There is evidence from the literature that SLAM may correlate with Th1 bias in T-cell cultures. Applicants therefore sought to determine whether combinations of Th1 or Th2 skewing cytokines might influence the expression of SLAM in TIL. To this end Applicants set up model rapid expansion protocols (REP) by mixing post-outgrowth TIL with mixed irradiated PBMC and phytohaemagglutinin in a G-rex plate. The relevant cytokines were added at day 0 and at time points during the expansion. After 14 days the cells were removed and stained to ascertain cell counts, by flow cytometry, as well as to determine the frequency of SLAM+, CD4+ and CD8+ cells, as well as CD45RO and CD62L+ cells as a marker of central memory (CM), effector memory (EM), Naïve-like (NL) and effector (E) cells (CM=CD45RO+/CD62L+; EM=CD45RO+/CD62L−; NL=CD45RO−/CD62L+; E=CD45RO−/CD62L−). Applicants chose the cytokines based on previous studies and observations. For example TL-4 is known as a potent driver of Th2 bias, whereas IL-12 drives Th1 bias (Heufler et al. 1996). Applicants also included IFN7 and IL-18 as there is evidence that these can also enhance Th1 skewing (Li et al. 2005; Smeltz et al. 2002). Finally, IL-7 and IL-15 were included in condition together as IL-7 can also potentiate TH1 bias (Lee et al. Sci Trans Med 2011) and IL-15 was added with IL-7 as this combination is generally used in combination (Gong et al. 2019; Zoon et al. 2014). Applicants included the cytokines with and without IL-2 which is normally added to TIL REPs, Applicants also included a control well of no cytokine. IL-4 was added at the beginning only for two donors and all the way through the culture for the other two. This was to ascertain whether kinetics of administration of this cytokine would influence phenotype.

Counts were made post REP to determine fold expansion of the TIL. Applicants found that no cytokine treatment induced the smallest overall expansion, IL-4, IL12, IFNγ and IL-18 alone were also less efficient at driving expansion, but IL-7 and IL-15 alone was similar to IL-2 alone, as was the combination of IL-2 with IL-4, IL-7+IL-15 and IL-18 and IFNγ (FIG. 8A). Expansion appeared to be limited by the combination of IL-2 and IL-12, an observation which may be due to the toxicity of IL-12 (Wang et al. 2017). The most striking differences were those which were mathematically significant i.e. untreated v IL-2, untreated v IL-7+IL-15, IFNγ v IL-7+IL-15 and untreated v IL-2+IFNγ (all p>0.05 by Friedman test).

The total frequency of CD4+ cells under each condition did not appear to differ greatly (FIG. 8C), however CD8+ frequency appeared to be decreased in IL-4 treated cells but increased in IL-2+IL-7+IL-15 and in IL-2+IL-12 treated cells (FIG. 8B). Applicants equally saw some interesting differences in the proportions of CM and EM cells. CM cells were enriched in cells treated with IL-12 or IL-2+IL-12, and lowest in IL-2+IL-4 (FIG. 8E). Conversely, the effect was seen with regards EM with the highest proportion in cells treated with IL-2+IL-4 and lowest in cells treated with IL-12 or IL-2+IL-12 (FIG. 8D).

When Applicants looked at SLAM expression it was found that in the CD4+ cells SLAM was highest in cells treated with IL-2+IL-7+IL-15, IL-2+IL-12 and IL-2+IL-18 (FIG. 9A). Applicants observed a significant difference between IFNγ v IL-2+IL-7+IL-15 treated cells. The effects of SLAM in CD4+ cells were far more evident in CM cells where conditions containing IL-4, IL-18 alone or IFNγ alone had lowest expression whereas highest expression was found in cells treated with IL-2+IL-7+IL-15 and IL-2+IL-12 (FIG. 9B). A significant difference between cells treated with IL-2+IL-12 v untreated was seen (p>0.05). In the EM cells the differences were smaller but a significant difference was seen between IL-12 v IL-2+IL-7+IL-15 (p>0.05) (FIG. 9C). SLAM expression in CD8+ cells generally mirrored CD4+ cells with lowest expression seen in cells treated with IL-4, however in CD8+ cells Applicants found that IL-2+IL-18 enhanced SLAM expression, particularly compared to IL-4 treated cells (p>0.05) (FIG. 9D). Akin to CD4+ cells the biggest difference was evident in the CM cells, however the observation made with IL-18 was also evident in the EM cells.

In summary from this experiment it appeared that IL-2, IL-12, IL-18 and IL-7+IL-15 were supportive of SLAM expression whereas IL-4 had the opposite effect. IL-12 however appeared to have some toxicity issues whereas IL-7+IL-15 looked interesting as they could mitigate the effects of low T-cell expansion as well as increasing SLAM expression.

Applicants therefore attempted a second experiment with some refined conditions. Applicants included IL-2 alone, IL-2+IL-12, IL-2+IL-4 as previous but now included additional conditions. IL-2+anti (α) IL-4 antibody, in an attempt to neutralise any free IL-4 which may have Th1 skewing capabilities, IL-2+IL-12+αIL-4, and then conditions in which IL-2 and IL-12 was added at the beginning of the culture, but then the IL-12 replaced with IL-2+IL-7+IL-15 for the remainder of the expansion, with or without the addition of anti-IL-4 (FIG. 10A). Overall expansion was relatively equivalent in all conditions except IL-2 or IL-2+IL-4. CD4+ frequency appeared to be favoured in IL-2+IL-4 v IL-2+IL-12+IL-7+IL-15±αIL-4 (all p>0.05) (FIG. 10B), whereas CD8+ frequency was lower in IL-2+IL-4 treated cells (FIG. 10C). Central memory cells were less favoured in the condition of IL-2+IL-4, with IL-2+IL-12±αIL-4 favouring their frequency (p>0.05) (FIG. 10D). EM was favoured in the condition of IL-2+IL-4 particularly with respect to IL-2+IL-12 (p>0.01) (FIG. 10E). When analysing SLAM Applicants found that CD4+ cells had lower SLAM in the IL-2+IL4 condition compared to IL-2+IL12+IL-7+IL-15+αIL-4 (p>0.05) (FIG. 11A), the same was equally true in the CD8+ cells although IL-2+IL-12 was significantly higher with regards SLAM than IL-2+IL-4 as well (p>0.05) (FIG. 11B). There was no obvious difference with regards SLAM expression in EM populations of CD4+(FIG. 11D) or CD8+(FIG. 11F), however in the CD4+(FIG. 11C) and CD8+(FIG. 11E) CM populations SLAM expression was significantly lower in IL-2+IL-4 than IL-2+IL-12+Il-7+IL-15+αIL-4.

Next Applicants investigated whether the conditions used to drive SLAM high and low phenotypes impacted on the ability of TIL to respond to mitogenic stimulation. To this end Applicants incubated TIL with K562 engineered to express a surface bound OKT3 single chain antibody fragment. After 6 h stimulation cells were permeabilised and intracellular staining for CD107a (a marker of degranulation), TNFα, IFNγ and IL-2 was performed with counterstaining for CD8 performed to gate on the CD8+ and CD8−, the latter which should be largely gating in the CD4+. The ability of CD8+ and CD8− cells to degranulate was largely unaffected by the cytokine cocktail used during the expansion phase, although in the CD8− both IL-2 alone and IL-2+IL-4 tended to reduce degranulation. Production of IL-2 by CD8+ cells was significantly worse in the presence of IL-12+anti-IL-4 compared to IL-2+anti-IL-4 (P>0.05), with IL-12 generally reducing IL-2 production but being rescued by the presence of IL-7 and IL-15 (FIG. 12B). The same observation was made in the CD8− cells with regards IL-12 being a negative impact on subsequent IL-2 production (FIG. 13B). TNFα production was optimal in cells incubated with IL-2+IL-12+IL-7+IL-15+anti-IL-4 particularly when compared with IL2+IL-4 (p>0.05) (FIG. 12C), TNFα production was not seemingly affected in CD8− cells (FIG. 13C). Finally IFNγ production by CD8+ cells was most negatively impacted by prior culture with IL-2+IL-4 particularly compared to IL-2+IL-12+anti-IL-4 treated cells (p>0.05), and IL-2+IL12+IL-7+IL-15 (p>0.05) treated cells (FIG. 12D).

In summary TH2 skewing conditions have a negative impact on many aspects of T-cell phenotype and activity from the perspective of adoptive cell therapy, generally resulting in more CD4+ cells, more EM cells and a lower response to polyclonal stimulation. Conversely combinations of TH1 skewing cytokines, with the addition of TH2 blocking reagents enhance features associated with adoptive T-cell therapy including increased CD8+ frequency, greater CM proportions and enhanced activity in response to polyclonal stimulation.

### Example 4

The impact of continuous administration of modulatory cytokines throughout the TIL growth process on phenotype and function was determined. TIL from nine donors were grown throughout the initial outgrowth and rapid expansion protocol in IL-2 (3000 IU/ml), IL-2 with an initial administration of IL-12 (25 ng/ml), or IL-2 with an initial administration of IL-12 (25 ng/ml) followed by a switch to IL-7 and IL-15 (both 10 ng/ml). Following TIL growth cells were phenotyped for presence of CD4/CD8 cells as well as central/effector memory. Incubation with a cocktail of IL-2, IL-12, IL-7 and IL-15 significantly increased the proportion of CD4+ cells as well as CD4+/CD8+ cells compared to IL-2 alone, and concurrently reduced the proportion of CD4−/CD8− cells (FIG. 14). Cocultures containing the cytokine IL-12, with or without IL-7/15 also significantly increased the proportion of central memory (CD45RO+/CD62L+) compared to cells cultured in IL-2 alone, whereas the combination of IL-2 and IL-12 significantly reduced the proportion of effector memory (CD45RO+/CD62L−) cells (FIG. 15).

Five of the donor TIL were then cocultured with OKT3 expressing K562 cells and the proportion of cells capable of eliciting various effector functions (IFNγ, IL-2, TNFα and CD107a) was assessed using flow cytometry. No difference was observed in the proportion of cells producing IFNγ, TNFα, IL-2 or CD107a within the CD8+ population (FIG. 16A). In the CD8− (mainly CD4+ cells), a decreased frequency of IL-2 and CD107a mobilisation was observed in cells cultured in IL-2, IL-12, IL-7 and IL-15 compared to IL-2 and IL-12 alone (FIG. 16B).

Three of the donor TIL were also cocultured with matched autologous tumour cell lines to assess the impact of culture conditions on responses to the matched tumour from the same patient. As expected the responses to the matched line were much lower compared to responses to K563-OKT3 which provides polyclonal stimulation, with CD107a being the most readily observed functional readout. No significant differences were seen between the cells grown in different culture conditions, except in CD8+ cells where cells grown in IL-2+IL-12 produced significantly more TNFα compared to those incubated with IL-2, IL-12, IL-7 and IL-15 (FIG. 17A).

### REFERENCES

Aspeslagh S, Postel-Vinay S, Rusakiewicz S, Soria J C, Zitvogel L, Marabelle A. Eur J Cancer. 2016 January; 52:50-66. Rationale for anti-OX40 cancer immunotherapy.
Browning M B, Woodliff J E, Konkol M C, Pati N T, Ghosh S, Truitt R L, Johnson B D. Cell Immunol. 2004 February; 227(2):129-39. The T cell activation marker CD150 can be used to identify alloantigen-activated CD4(+)25+ regulatory T cells.
Cieri N, Camisa B, Cocchiarella F, Forcato M, Oliveira G, Provasi E, Bondanza A, Bordignon C, Peccatori J, Ciceri F, Lupo-Stanghellini M T, Mavilio F, Mondino A, Bicciato S, Recchia A, Bonini C. IL-7 and IL-15 instruct the generation of human memory stem T cells from naive precursors. Blood. 2013 Jan. 24; 121(4):573-84. doi: 10.1182/blood-2012-05-431718. Epub 2012 Nov. 15.
Cui G, Staron M M, Gray S M, Ho P C, Amezquita R A, Wu J, Kaech S M. IL-7-Induced Glycerol Transport and TAG Synthesis Promotes Memory CD8+ T Cell Longevity. Cell. 2015 May 7; 161(4):750-61. doi: 10.1016/j.cell.2015.03.021.
Dudley M E, Wunderlich J R, Shelton T E, Even J, Rosenberg S A. J Immunother. 2003 July-August; 26(4):332-42. Generation of tumor-infiltrating lymphocyte cultures for use in adoptive transfer therapy for melanoma patients.
Dudley M E, Gross C A, Langhan M M, Garcia M R, Sherry R M, Yang J C, Phan G Q, Kammula U S, Hughes M S, Citrin D E, Restifo N P, Wunderlich J R, Prieto P A, Hong J J, Langan R C, Zlott D A, Morton K E, White D E, Laurencot C M, Rosenberg S A. Clin Cancer Res. 2010 Dec. 15; 16(24):6122-31. CD8+ enriched “young” tumor infiltrating lymphocytes can mediate regression of metastatic melanoma.
Eisenhauer E A, Therasse P, Bogaerts J, Schwartz L H, Sargent D, Ford R, Dancey J, Arbuck S, Gwyther S, Mooney M, Rubinstein L, Shankar L, Dodd L, Kaplan R, Lacombe D, Verweij J. Eur J Cancer. 2009 January; 45(2):228-47 New response evaluation criteria in solid tumours: revised RECIST guideline (version 1.1).
Erlenhoefer C, Wurzer W J, Loffler S, Schneider-Schaulies S, ter Meulen V, Schneider-Schaulies J. J Virol. 2001. 75(10):4499-505 CD150 (SLAM) is a receptor for measles virus but is not involved in viral contact-mediated proliferation inhibition. J Virol. 2001 May;
Figlin R A, Pierce W C, Kaboo R, Tso C L, Moldawer N, Gitlitz B, deKernion J, Belldegrun A. J Urol. 1997 September; 158(3 Pt 1):740-5. Treatment of metastatic renal cell carcinoma with nephrectomy, interleukin-2 and cytokine-primed or CD8(+) selected tumor infiltrating lymphocytes from primary tumor.
Frecha C, Levy C, Costa C, Negre D, Amirache F, Buckland R, Russell S J, Cosset F L, Verhoeyen E. J Virol. 2011 June; 85(12):5975-85. Measles virus glycoprotein-pseudotyped lentiviral vector-mediated gene transfer into quiescent lymphocytes requires binding to both SLAM and CD46 entry receptors.
Gardini A, Ercolani G, Riccobon A, Ravaioli M, Ridolfi L, Flamini E, Ridolfi R, Grazi G L, Cavallari A, Amadori D. J Surg Oncol. 2004 Jul. 15; 87(1):46-52. Adjuvant, adoptive immunotherapy with tumor infiltrating lymphocytes plus interleukin-2 after radical hepatic 30 resection for colorectal liver metastases: 5-year analysis.
Goedegebuure P S, Douville L M, Li H, Richmond G C, Schoof D D, Scavone M, Eberlein T J. J Clin Oncol. 1995 August; 13(8):1939-49. Adoptive immunotherapy with tumor-infiltrating lymphocytes and interleukin-2 in patients with metastatic malignant melanoma and renal cell carcinoma: a pilot study.
Gong W, Hoffmann J M, Stock S, Wang L, Liu Y, Schubert M L, Neuber B, Huckelhoven-Krauss A, Gem U, Schmitt A, Müller-Tidow C, Shiku H, Schmitt M, Sellner L. Comparison of IL-2 vs IL-7/IL-15 for the generation of NY-ESO-1-specific T cells. Cancer Immunol Immunother. 2019 July; 68(7):1195-1209. doi: 10.1007/s00262-019-02354-4. Epub 2019 Jun. 8.
Grupp S A, Kalos M, Barrett D, Aplenc R, Porter D L, Rheingold S R, Teachey D T, Chew A, Hauck B, Wright J F, Milone M C, Levine B L, June C H. N Engl J Med. 2013 Apr. 18; 368(16):1509-18. Chimeric antigen receptor-modified T cells for acute lymphoid leukemia.
Hamalainen H, Meissner S, Lahesmaa R. J Immunol Methods. 2000 Aug. 28; 242(1-2):9-19. Signaling lymphocytic activation molecule (SLAM) is differentially expressed in human Th1 and Th2 cells.
Harrop R, Treasure P, de Belin J, Kelleher M, Bolton G, Naylor S, Shingler W H. Cancer Immunol Immunother. 2012 December; 61(12):2283-94. Analysis of pre-treatment markers predictive of treatment benefit for the therapeutic cancer vaccine MVA-5T4 (TroVax).
Haymaker C L, Wu R C, Ritthipichai K, Bernatchez C, Forget M A, Chen J Q, Liu H, Wang E, Marincola F, Hwu P, Radvanyi L G. Oncoimmunology. 2015 Mar. 16; 4(8): BTLA marks a less-differentiated tumor-infiltrating lymphocyte subset in melanoma with enhanced survival properties.
Heufler C, Koch F, Stanzl U, Topar G, Wysocka M, Trinchieri G, Enk A, Steinman R M, Romani N, Schuler G. Eur J Immunol. 1996 March; 26(3):659-68. Interleukin-12 is produced by dendritic cells and mediates T helper 1 development as well as interferon-gamma production by T helper 1 cells.
Knee D A, Hewes B, Brogdon J L. Eur J Cancer. 2016 November; 67:1-10. Rationale for anti-GITR cancer immunotherapy.
Kochenderfer J N, Dudley M E, Kassim S H, Somerville R P, Carpenter R O, Stetler-Stevenson M, Yang J C, Phan G Q, Hughes M S, Sherry R M, Raffeld M, Feldman S, Lu L, Li Y F, Ngo L T, GoyA, Feldman T, Spaner D E, Wang M L, Chen C C, Kranick S M, Nath A, Nathan D A, Morton K E, Toomey M A, Rosenberg S A. J Clin Oncol. 2015 33(6):540-9. Chemotherapy-refractory diffuse large B-cell lymphoma and indolent B-cell malignancies can be effectively treated with autologous T cells expressing an anti-CD19 chimeric antigen receptor.
Mehrle S, Schmidt J, Büchler MW, Watzl C, Marten A. Mol Immunol. 2008 February; 45(3):796-804. Enhancement of anti-tumor activity in vitro and in vivo by CD150 and SAP.
Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. Science. 2006 Oct. 6; 314(5796):126-9. Cancer regression in patients after transfer of genetically engineered lymphocytes.
Lee L F, Axtell R, Tu G H, Logronio K, Dilley J, Yu J, Rickert M, Han B, Evering W, Walker M G, Shi J, de Jong B A, Killestein J, Polman C H, Steinman L, Lin J C. Sci Transl Med. 2011 Jul. 27; 3(93). IL-7 promotes T(H)1 development and serum IL-7 predicts clinical response to interferon-0 in multiple sclerosis.
Li Q, Carr A L, Donald E J, Skitzki J J, Okuyama R, Stoolman L M, Chang A E. Cancer Res. 2005 Feb. 1; 65(3):1063-70. Synergistic effects of IL-12 and IL-18 in skewing tumor-reactive T-cell responses towards a type 1 pattern.
Pegram H J, Purdon T J, van Leeuwen D G, Curran K J, Giralt S A, Barker J N, Brentjens R J. IL-12-secreting CD19-targeted cord blood-derived T cells for the immunotherapy of B-cell acute lymphoblastic leukemia. Leukemia. 2015 February; 29(2):415-22. doi: 10.1038/leu.2014.215. Epub 2014 Jul. 9.
Petty J K, He K, Corless C L, Vetto J T, Weinberg A D. Am J Surg. 2002 May; 183(5):512-8. Survival in human colorectal cancer correlates with expression of the T-cell costimulatory 30 molecule OX-40 (CD134).
Quiroga M F, Martinez G J, Pasquinelli V, Costas M A, Bracco M M, Malbrin A, Olivares L M, Sieling P A, Garcia V E. J Immunol. 2004 Sep. 15; 173(6):4120-9. Activation of signaling lymphocytic activation molecule triggers a signaling cascade that enhances Th1 responses in human intracellular infection.
Radvanyi L G, Bernatchez C, Zhang M, Fox P S, Miller P, Chacon J, Wu R, Lizee G, Mahoney S, Alvarado G, Glass M, Johnson V E, McMannis J D, Shpall E, Prieto V, Papadopoulos N, Kim K, Homsi J, Bedikian A, Hwu W J, Patel S, Ross M I, Lee J E, Gershenwald J E, Lucci A, Royal R, Cormier J N, Davies M A, Mansaray R, Fulbright O J, Toth C, Ramachandran R, Wardell S, Gonzalez A, Hwu P. Clin Cancer Res. 2012 Dec. 15; 18(24):6758-70. Specific lymphocyte subsets predict response to adoptive cell therapy using expanded autologous tumor-infiltrating lymphocytes in metastatic melanoma patients.
Rapoport A P, Stadtmauer E A, Binder-Scholl G K, Goloubeva O, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin H K, Ribeiro L, Holdich T, Yanovich S, Hardy N, Yared J, Kerr N, Philip S, Westphal S, Siegel D L, Levine B L, Jakobsen B K, Kalos M, June C H. Nat Med. 2015 August; 21(8):914-21. NY-ESO-1-specific TCR-engineered T cells mediate sustained antigen-specific antitumor effects in myeloma.
Rosenberg S A, Yang J C, Sherry R M, Kammula U S, Hughes M S, Phan G Q, Citrin D E, Restifo N P, Robbins P F, Wunderlich J R, Morton K E, Laurencot C M, Steinberg S M, White D E, Dudley M E. Clin Cancer Res. 2011 Jul. 1; 17(13):4550-7. Durable complete responses in heavily pretreated patients with metastatic melanoma using T-cell transfer immunotherapy.
So T, Croft M. J Immunol. 2007. Cutting edge: OX40 inhibits TGF-beta- and antigen-driven conversion of naive CD4 T cells into CD25+Foxp3+ T cells.
Shimizu J, Yamazaki S, Takahashi T, Ishida Y, Sakaguchi S. Nat Immunol. 2002 February; 3(2):135-42. Stimulation of CD25(+)CD4(+) regulatory T cells through GITR breaks immunological self-tolerance.
Schwartz L H, Litiere S, de Vries E, et al. RECIST 1.1-Update and clarification: From the RECIST committee. Eur J Cancer. 2016; 62:132-7.
Stevanovid S, Draper L M, Langhan M M, Campbell T E, Kwong M L, Wunderlich J R, Dudley M E, Yang J C, Sherry R M, Kammula U S, Restifo N P, Rosenberg S A, Hinrichs C S. J Clin Oncol. 2015 May 10; 33(14):1543-50. Complete regression of metastatic cervical cancer after treatment with human papillomavirus-targeted tumor-infiltrating T cells.
Topalian S L, Hodi F S, Brahmer J R, Gettinger S N, Smith D C, McDermott D F, Powderly J D, Carvajal R D, Sosman J A, Atkins M B, Leming P D, Spigel D R, Antonia S J, Horn L, Drake C G, Pardoll D M, Chen L, Sharfman W H, Anders R A, Taube J M, McMiller T L, Xu H, Korman A J, Jure-Kunkel M, Agrawal S, McDonald D, Kollia G D, Gupta A, Wigginton J M, Sznol M. N Engl J Med. 2012 Jun. 28; 366(26):2443-54. Safety, activity, and immune correlates of anti-PD-1 antibody in cancer.
Tran E, Turcotte S, Gros A, Robbins P F, Lu Y C, Dudley M E, Wunderlich J R, Somerville R P, Hogan K, Hinrichs C S, Parkhurst M R, Yang J C, Rosenberg S A. Science. 2014 May 9; 344(6184):641-5. Cancer immunotherapy based on mutation-specific CD4+ T cells in a patient with epithelial cancer.
van Olffen R W, Koning N, van Gisbergen K P, Wensveen F M, Hoek R M, Boon L, Hamann J, van Lier R A, Nolte M A. J Immunol. 2009 Jun. 15; 182(12):7490-500. GITR triggering induces expansion of both effector and regulatory CD4+ T cells in vivo.
Vu M D, Xiao X, Gao W, Degauque N, Chen M, Kroemer A, Killeen N, Ishii N, Li X C. Blood. 2007. OX40 costimulation turns off Foxp3+ Tregs.
Wang P, Li X, Wang J, Gao D, Li Y, Li H, Chu Y, Zhang Z, Liu H, Jiang G, Cheng Z, Wang S, Dong J, Feng B, Chard L S, Lemoine N R, Wang Y. Nat Commun. 2017 Nov. 9; 8(1):1395. Re-designing Interleukin-12 to enhance its safety and potential as an anti-tumor immunotherapeutic agent.
Watanabe N, Gavrieli M, Sedy J R, Yang J, Fallarino F, Loftin S K, Hurchla M A, Zimmerman N, Sim J, Zang X, Murphy T L, Russell J H, Allison J P, Murphy K M. Nat Immunol. 2003 July; 4(7):670-9. BTLA is a lymphocyte inhibitory receptor with similarities to CTLA-4 and PD-1.
Xu X, Xu L, Ding S, Wu M, Tang Z, Fu W, Ni Q. Chin Med Sci J. 1995 September; 10(3):185-7. Treatment of 23 patients with advanced gastric cancer by intravenously transfer of autologous tumor-infiltrating lymphocytes combined with rIL-2.
Xu Y, Zhang M, Ramos C A, Durett A, Liu E, Dakhova O, Liu H, Creighton C J, Gee A P, Heslop H E, Rooney C M, Savoldo B, Dotti G. Closely related T-memory stem cells correlate with in vivo expansion of CAR.CD19-T cells and are preserved by IL-7 and IL-15. Blood. 2014 Jun. 12; 123(24):3750-9. doi: 10.1182/blood-2014-01-552174. Epub 2014 Apr. 29.
Zhang L, Kerkar S P, Yu Z, Zheng Z, Yang S, Restifo N P, Rosenberg S A, Morgan R A. Improving adoptive T cell therapy by targeting and controlling IL-12 expression to the tumor environment. Mol Ther. 2011 April; 19(4):751-9. doi: 10.1038/mt.2010.313. Epub 2011 Feb. 1.
Zoon C K, Seitelman E, Keller S, Graham L, Blevins T L, Dumur C I, Bear H D. Cancer Gene Ther. 2014 October; 21(10):441-7. doi: 10.1038/cgt.2014.48. Epub 2014 Sep. 19. Expansion of melanoma-specific lymphocytes in alternate gamma chain cytokines: gene expression variances between T cells and T-cell subsets exposed to IL-2 versus IL-7/15.

Aspects and embodiments of the invention are also set out in the following clauses:

1. An in vitro method for the prognosis of patients undergoing tumour infiltrating lymphocyte (TIL) therapy which method comprises the following steps:

a. quantifying, in a sample of TIL from said patient, a biological marker (CD150/SLAM/SLAMF1) indicative of the status of the adaptive immune response; and

b. said biological marker (CD150/SLAM/SLAMF1) is indicative of the cancer response; and

c. Comparing the value obtained at step a) for said at least one biological marker (CD150/SLAM/SLAMF1) with a predetermined reference value for the same biological marker; for which there is a predetermined reference value that correlates with a specific prognosis or progression of said cancer.

2. The in vitro method according to clause 1, wherein said tumour tissue sample originates from the group consisting of (i) a primary tumour, (ii) a metastatic tumour lesion, (iii) the lymph nodes located at the closest proximity to one of the tumour lesions from said patient.
3. The in vitro method according to clause 1, wherein step a) is performed using flow cytometry.
4. The in vitro method according to clause 1, wherein step a) is performed using an alternative direct or indirect method of assessing the value of said biological marker (CD150/SLAM/SLAMF1) and said method comparing the value obtained at step a) for said at least one biological marker (CD150/SLAM/SLAMF1) enables determination of a predetermined reference value for the same biological marker; for which there is a predetermined reference value that correlates with a specific prognosis or progression of said cancer.
5. The in vitro method according to clause 1, wherein said biological marker (CD150/SLAM/SLAMF1) is expressed by a lymphocyte.
6. The in vitro method according to clause 1, wherein said biological marker (CD150/SLAM/SLAMF1) indicative of the status of the adaptive immune response of said patient against said patients cancer consists of at least one biological marker expressed by a cell from the immune system selected from the group consisting of T lymphocytes, NK cells, NKT cells, γδ T-cells, CD4+ cells and/or CD8+ cells.
7. The in vitro method according to clause 1, wherein said biological marker is SLAM (CD150).
8. The in vitro method according to clause 1, wherein said biological marker (CD150/SLAM/SLAMF1) is quantified in one of the following samples: (i) a single cell suspension of tumour, (ii) a sample of material obtained from the single cell suspension of tumour at any point during the culture of tumour infiltrating lymphocytes, (iii) the final product to be issued for infusion to said patient.
9. An in vitro method of selecting cells expressing prognostically favourable levels of said biological marker (CD150/SLAM/SLAMF1) using one or more of the following selection techniques: (i) flow cytometry, (ii) antibody panning, (iii) magnetic selection, (iv) biomarker targeted cell enrichment.
10. The in vitro method according to clause 9, wherein the cells expressing the said biological marker (CD150/SLAM/SLAMF1) are expanded by way of one or both of the following options:

c) Irradiated feeder cells in such a fashion as to provide a T-cell activation signal and costimulation driven by antibodies or costimulatory receptors; and

d) Immobilised or soluble reagents which provide a T-cell activation signal and costimulation signals driven through said one or more biological markers.

11. An in vitro method of expressing said biological marker (CD150/SLAM/SLAMF1) in one or more lymphocytes.
12. A vector which comprises a nucleic acid sequence according to clause 11.
13. A cell which expresses a polypeptide according to clause 11.
14. A method for making a cell according to clause 11 which comprises the step of transducing or transfecting a cell with a vector according to any of clauses 12.
15. A vector according to clause 12, wherein the transgene of interest also encodes a chimeric antigen receptor, a T-cell receptor or another receptor of immunotherapeutic use for adoptive cell therapy, such that when the vector is used to transduce a target cell, the target cell co-expresses a polypeptide according to any of clause 13 and a chimeric antigen receptor, T-cell receptor or another receptor of immunotherapeutic interest. For clarity this additional polypeptide encoded protein is termed a protein of interest (POI)
16. A method for selecting cells expressing a POI which comprises the following steps:

I. detecting expression of the POI epitope on the surface of cells transfected or transduced with a vector according to clause 15; and

II. Enriching for cells which are identified as expressing the POI epitope.

17. A method for preparing a purified population of cells enriched for cells expressing a POI which comprises the step of selecting cells expressing a POI from a population of cells using a method according to clause 16.
18. A cell population which is enriched for cells expressing a polypeptide according to clause 1, and thus enriched for cells expressing a POI.
19. A method for tracking transduced cells in vivo which comprises the step of detection of expression of a polypeptide according to clause 1 at the cell surface.
20. A method for treating a disease in a subject, which comprises the step of administering a cell according to any of clauses 12-14, or a cell population according to clause 18 to the subject.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for preparing an enriched and expanded cell population of tumour reactive T-cells for use in cancer therapy comprising identifying and/or obtaining a cell population expressing CD150 and expanding the cell population.

2. The method of claim 1, wherein the T-cells expressing CD150 are T-cells selected from cells originating from a subject.

3. The method of claim 1, wherein selecting the T-cells expressing CD150 comprises contacting the cell population with an anti-CD150 antibody or comprises one or more of (i) flow cytometry, (ii) antibody panning, (iii) magnetic selection, and (iv) biomarker targeted cell enrichment.

4. (canceled)

5. The method of claim 1, wherein the expanding comprises:
(a) irradiating feeder cells and co-stimulating with an anti-CD150 antibody and optionally one or more cytokines;
(b) stimulating or activating CD150; or
(c) rapid expansion protocol (REP) wherein cells are mixed with irradiated feeder cells and one or more cytokines until at least 1×10⁹ or at least 5×10⁹ or at least 1×10¹⁰ cells are obtained.

6. The method of claim 1, wherein the cell population is:
(a) a population of tumour infiltrating lymphocytes (TILs) from a tumour biopsy, lymph node, or ascites;
(b) a population of T-cells engineered to express a CAR and/or a TCR; and/or
(c) a population of T-cells isolated from blood.

7. The method of claim 1, wherein the cancer therapy is adoptive cell therapy and/or wherein the cancer is a melanoma, lung cancer, squamous cell cancer, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, head cancer or neck cancer.

8. (canceled)

9. The method of claim 1, wherein the method further comprises exposing the T-cells to a modulator that increases CD150 production.

10. The method of claim 9, wherein the modulator comprises:
(I) a cytokine, wherein the cytokine is IL-2, IL-15, IL-18, IL-12, IL-23, IL-27, IL-35, IL-39, IL-18, IL-36, IL-37, IL-38, IFN-alpha, IFN-beta, IFN-gamma, or a combination thereof; or
(II) a cytokine combination of IL-7+IL-15, IL-2+IL-7+IL-15, IL-2+IL-12, IL-2+IL-18, IL-2+IL-12+IL-7+IL-15, IL-2+IL-12+IL-7+IL-15+IL-6, IL-2+IL-12+IL-7+IL-15+IL-21, IL-2+IL-12+IL-7+IL-15+IL-6+IL-21, IL-7+IL-15+IL-6, IL-7+IL-15+IL-21, IL-7+IL-15+IL-6+IL-21, IL-2+IL-12+IL-6, IL-2+IL-12+IL-21, or IL-2+IL-12+IL-6+IL-21.

11. (canceled)

12. (canceled)

13. The method of claim 9, wherein the modulator further comprises a Th2 blocking agent, wherein the Th2 blocking agent is an antibody,
(I) wherein the antibody is selected from anti-IL-4 (αIL4), anti-IL-4R (αIL4R), anti-IL-5R (αIL5R), anti-IL-5 (αIL5), anti-IL-13R (αIL13R), and anti-IL-13 (αIL13); or
(II) wherein the antibody is selected from Mepolizumab, Resilizumab, Benralizumab, Tralokinumab, Lebrikizumab, and Dupilumab.

14.-16. (canceled)

17. The method of claim 13, wherein the modulator comprises:
(I) IL-2+αIL4;
(II) IL-2+IL-12+αIL4; or
(III) IL-2+IL-12+IL-7+IL-15+αIL4.

18. (canceled)

19. (canceled)

20. The method of claim 10, wherein IL-12 is briefly added for 1-96 h at 1-150 ng/ml at the expanding step and subsequently replaced with another cytokine, and/or wherein the cytokine is produced by an artificial antigen presenting cell and the tumour reactive T-cells are co-cultured with the antigen presenting cell.

21. (canceled)

22. The method of claim 1, wherein the cell population is isolated; and cultured with a modulator that increases CD150 production, and wherein the cultured cells are separated from unselected cells.

23. The method of claim 1, wherein the T-cells are engineered to express a cytokine receptor which provides cytokine signaling upon engagement of another drug or cytokine, and/or wherein the T-cell is a CD4+ or CD8+ T-cell.

24. (canceled)

25. A population of cells obtained according to the method of claim 1.

26. (canceled)

27. (canceled)

28. The population of cells of claim 25, wherein the population comprises T-cells, and wherein >25%, >30%, or >40% of the T-cells express CD150.

29. The population of cells of claim 25, wherein the cells are TILs.

30. (canceled)

31. (canceled)

32. A pharmaceutical composition comprising the population of cells of claim 25.

33. (canceled)

34. A method for assessing the tumour reactivity of a cell population, comprising quantifying the proportion of T-cells expressing CD150 in the cell population.

35. The method of claim 34 wherein the cell population comprises:
(I) TILs from a patient, and the T-cells expressing CD150 are quantified pre-rapid expansion protocol (REP) and/or post-REP; or
(II) a population of T-cells engineered to express an exogenous CAR and/or a TCR.

36. The method of claim 34, wherein the proportion of T-cells expressing CD150 being at least 25% of the cell population indicates that the cell population is tumour reactive.

37. A method for identifying an agent for increasing CD150 expression in isolated ex vivo expansion of T-cells for use in adoptive cell therapy, comprising:
(a) contacting tumour infiltrating lymphocytes with a candidate modulator that upregulates the expression of CD150 in T-cells;
(b) screening the effect of the modulator on the expression of CD150 in the cells; and
(c) identifying a modulator that increases the expression of CD150 in the cells.

38. The method of claim 37, wherein the T-cell is a CD4+ or a CD8+ T-cell.

39. An in vitro method for the prognosis of a patient who may receive tumour infiltrating lymphocyte (TIL) therapy for cancer, comprising:
(a) quantifying in a sample of tumour digest, TIL material during the manufacturing process or TIL product, from the patient, at least one biological marker on the TIL; and
(b) comparing the value obtained at step (a) for the at least one biological marker with a predetermined reference value for the same biological marker; which predetermined reference value is correlated with a specific prognosis of progression of the cancer, wherein the at least one biological marker is CD150.

40. (canceled)

41. A method of cancer therapy, comprising preparing an enriched and expanded cell population of tumour reactive T-cells, comprising identifying and/or obtaining a cell population expressing CD150, expanding the cell population, and administering to a cancer patient in need thereof cells from the cell population.

42. The method of claim 41, wherein the T-cells expressing CD150 are T-cells selected from cells originating from a subject.

43. The method of claim 41, wherein selecting the T-cells expressing CD150 comprises contacting the cell population with an anti-CD150 antibody or comprises one or more of (i) flow cytometry, (ii) antibody panning, (iii) magnetic selection, and (iv) biomarker targeted cell enrichment.

44. (canceled)

45. The method of claim 41, wherein the expanding comprises:
(a) irradiating feeder cells and co-stimulating with an anti-CD150 antibody and optionally one or more cytokines;
(b) stimulating or activating CD150; or
(c) rapid expansion protocol (REP), wherein cells are mixed with irradiated feeder cells and one or more cytokines until at least 1×10⁹ or at least 5×10⁹ or at least 1×10¹⁰ cells are obtained.

46. The method of claim 41, wherein the cell population is:
(a) a population of tumour infiltrating lymphocytes (TILs) from a tumour biopsy, lymph node, or ascites;
(b) a population of T-cells engineered to express a CAR and/or a TCR; and/or
(c) a population of T-cells isolated from blood.

47. The method of claim 41, wherein the cancer therapy is adoptive cell therapy and/or wherein the cancer is a melanoma, lung cancer, or ovarian cancer.

48. (canceled)

49. The method of claim 41, wherein the method further comprises exposing the T-cells to a modulator that increases CD150 production.

50. The method of claim 49, wherein the modulator comprises:
(I) a cytokine, wherein the cytokine is IL-2, IL-15, IL-18, IL-12, IL-23, IL-27, IL-35, IL-39, IL-18, IL-36, IL-37, IL-38, IFN-alpha, IFN-beta, IFN-gamma, or a combination thereof; or
(II) a cytokine combination of IL-7+IL-15, IL-2+IL-7+IL-15, IL-2+IL-12, IL-2+IL-18, IL-2+IL-12+IL-7+IL-15, IL-2+IL-12+IL-7+IL-15+IL-6, IL-2+IL-12+IL-7+IL-15+IL-21, IL-2+IL-12+IL-7+IL-15+IL-6+IL-21, IL-7+IL-15+IL-6, IL-7+IL-15+IL-21, IL-7+IL-15+IL-6+IL-21, IL-2+IL-12+IL-6, IL-2+IL-12+IL-21, or IL-2+IL-12+IL-6+IL-21.

51. (canceled)

52. (canceled)

53. The method of claim 49, wherein the modulator further comprises a Th2 blocking agent, wherein the Th2 blocking agent is an antibody,
(I) wherein the antibody is selected from anti-IL-4 (αIL4), anti-IL-4R (αIL4R), anti-IL-5R (αIL5R), anti-IL-5 (αIL5), anti-IL-13R (αIL13R), and anti-IL-13 (αIL13); or
(II) wherein the antibody is selected from Mepolizumab, Resilizumab, Benralizumab, Tralokinumab, Lebrikizumab, and Dupilumab.

54.-56. (canceled)

57. The method of claim 53, wherein the modulator comprises:
(I) IL-2+αIL4;
(II) IL-2+IL-12+αIL4; or
(III) IL-2+IL-12+IL-7+IL-15+αIL4.

58. (canceled)

59. (canceled)

60. The method of claim 50, wherein IL-12 is briefly added for 1-96 h at 1-150 ng/ml at the expanding step and subsequently replaced with another cytokine, and/or wherein the cytokine is produced by an artificial antigen presenting cell and the tumour reactive T-cells are co-cultured with the antigen presenting cell.

61. (canceled)

62. The method of claim 41, wherein the cell population is isolated- and cultured with a modulator that increases CD150 production, and the cultured cells are separated from unselected cells, and/or wherein the T-cells are engineered to express a cytokine receptor which provides cytokine signaling upon engagement of another drug or cytokine.

63.-89. (canceled)

90. A method for increasing expression of CD150 in tumour reactive T-cells for use in cancer therapy, comprising identifying and/or obtaining a cell population expressing CD150 and expanding the cell population in the presence of a cytokine that increases expression of CD150.

91. The method of claim 90, wherein the cytokine is used in combination with a Th2 blocking agent.
